(19) United States
(12) Patent Application Publication
(10) Pub. No.: US 2021/0324366 A1
(43) Pub. Date: Oct. 21, 2021

(54) SUPPORTED PLANT PLASMA MEMBRANE LIPID BILAYER ON-A-CHIP

(71) Applicant: Cornell University, Ithaca, NY (US)

(72) Inventors: Susan DANIEL, Ithaca, NY (US); Martin STUEBLER, Ithaca, NY (US); Han-Yuan LIU, Ithaca, NY (US)

(21) Appl. No.: 17/258,863

(22) PCT Filed: Jul. 11, 2019
(86) PCT No.: PCT/US19/41402

§ 371 Date: Jan. 8, 2021

Related U.S. Application Data
(60) Provisional application No. 62/696,424, filed on Jul. 11, 2018.

(51) Int. Cl. C12N 11/08

(52) U.S. Cl. CPC C12N 11/08

(57) ABSTRACT

Provided herein are compositions comprising a plant plasma membrane supported on a surface of an object, wherein the plant plasma membrane is not associated with a plant cell.

### CROSS-REFERENCE TO RELATED APPLICATIONS

This application claims benefit of U.S. Provisional Application No. 62/696,424, filed Jul. 11, 2018, the contents of which are incorporated herein by reference in their entirety.

### GOVERNMENT FUNDING

This invention was made with Government support under Grant Number CBET-1149452 and CBET-1263701 awarded by the National Science Foundation. The United States Government has certain rights in the invention.

### BACKGROUND

The following description is provided to assist the understanding of the reader. None of the information provided or references cited is admitted to be prior art to the present technology.

Plant plasma membranes are the site of useful and essential biological processes. These processes include ion channel function, plant enzyme function, light reactive centers, and so on. However, there is currently no system that allows the study of these processes or the proteins involved in their native plant membrane lipids in an in vitro platform that is compatible with various surface analytical tools and assays. As protein function often depends on the surrounding lipids and membrane properties, the absence of a means to study these proteins in a plant plasma membrane is severely limiting. Furthermore, such a platform would open the possibility to study other aspects of plant membrane function and stability, for example, pathogen interaction with the plant membrane, the impact of chemicals like herbicides, fertilizers, etc. on the membrane stability and mechanical properties, and the study of basic processes like the fusion of two plant membranes in the process of protoplast fusion.

### SUMMARY OF THE INVENTION

Disclosed herein are compositions comprising a plant plasma membrane supported on a surface of an object, wherein the plant plasma membrane is not associated with a plant cell, and methods for making supported plant plasma membranes.

In one aspect, provided herein is a composition comprising a plant plasma membrane supported on a surface of an object, wherein the plant plasma membrane is not associated with a plant cell.

In some embodiments, the plant plasma membrane is a lipid bilayer.

In some embodiments, the plant plasma membrane is adsorbed to the surface of the object. In other embodiments, the plant plasma membrane adheres to the surface of the object.

In some embodiments, the plant plasma membrane is derived from a cell plasma membrane. In some embodiments, the plant plasma membrane is derived from a chloroplast membrane, e.g., a thylakoid membrane.

In some embodiments, the plant plasma membrane is derived from a leaf, stem, or root of a plant. In some embodiments, the plant is a maize plant, an Arabidopsis plant, or a tobacco plant.

In some embodiments, the plant plasma membrane has a protein and lipid orientation, mobility, and activity that is native to the plant plasma membrane.

In some embodiments, the plant plasma membrane is modified. In some embodiments, the plant plasma membrane is derived from a plant or plant part that has been modified. In some embodiments, the modification comprises addition of a membrane component, e.g., a lipid or an exogenous protein (e.g., an exogenous protein comprising a fluorescent marker).

In some embodiments, the plant plasma membrane is produced by a method comprising blebbing.

In some embodiments, the plant plasma membrane is produced by a method comprising bubbles.

In some embodiments, the object is a glass microscope slide, a silicon wafer, a microfluidic channel, or a silica bead.

In some embodiments, the surface is hydrophilic. In some embodiments, the surface comprises a coating, e.g., a polymer, e.g., PMETAC. In some embodiments, the surface is chemically modified.

In some embodiments, the surface of the object is flat. In other embodiments, the surface of the object is round, curved, corrugated, or possesses another geometric shape or topography.

In a second aspect, provided herein is a method for manufacturing a plant plasma membrane supported on a surface of an object, the method comprising (a) providing a plurality of vesicles, wherein each vesicle comprises an isolated plant plasma membrane; and (b) contacting the plurality of vesicles with a surface of an object, wherein the isolated plant plasma membranes assemble into a continuous lipid bilayer, thereby forming the plant plasma membrane supported on the surface of the object.

In some embodiments, the plurality of vesicles is a plurality of bubbles and the providing comprises treating a plant or plant part to produce a plurality of bubbles. In some embodiments, the treating comprises administering an agent that disrupts a cell wall of the plant or plant part, e.g., a cellulase or a pectinase. In some embodiments, the agent comprises a cellulase and a pectinase.

In some embodiments of the above methods, the plant or plant part is a maize plant or plant part, an Arabidopsis plant or plant part, or a tobacco plant or plant part. In some embodiments, the plant part is a leaf, stem, or root of a plant.

In some embodiments, the plurality of vesicles is a plurality of blebs and the providing comprises (a) providing a plant protoplast; and (b) treating the plant protoplast to produce a plurality of blebs. In some embodiments, the providing of step (a) comprises administering an agent that disrupts a cell wall of the plant or plant part to produce a protoplast. In some embodiments, the treating of step (b) comprises administering a blebbing buffer. In some embodiments, the treating of step (b) comprises inducing osmotic stress.

In some embodiments of the above methods, the plant protoplast is a maize protoplast, an Arabidopsis protoplast, or a tobacco protoplast.

In some embodiments, the plurality of vesicles is a plurality of bubbles and blebs and the providing comprises (a) treating a plant or plant part to produce a plurality of bubbles; (b) treating the plant or plant part of step (a) to produce a plant protoplast; and (c) treating the plant protoplast of step (b) to produce a plurality of blebs.

In some embodiments of the above methods, the plant, plant part, or plant protoplast is a maize plant, plant part, or protoplast, an Arabidopsis plant, plant part, or protoplast, or a tobacco plant, plant part, or protoplast. In some embodiments, the plant part is a leaf, stem, or root of a plant.

In some embodiments, the plant plasma membrane is a lipid bilayer. In some embodiments, the plasma membrane is derived from a cell plasma membrane.

In some embodiments, the plant part is a chloroplast. In some embodiments, the plasma membrane is a chloroplast membrane, e.g., a thylakoid membrane.

In some embodiments of the above methods, the method further comprises modifying the plant plasma membrane.

In some embodiments of the above methods, the contacting comprises causing the plurality of vesicles to rupture on a surface, wherein the ruptured vesicles create a continuous lipid bilayer. In some embodiments, causing the plurality of vesicles to rupture comprises providing a surface comprising one or more attractive moieties. In some embodiments, the attractive moiety is comprised by a coating on the surface, e.g., a polymer coating, e.g., PMETAC. In some embodiments, the attractive moiety is comprised by a chemical modification on the surface.

In some embodiments of the above methods, the plurality of vesicles rupture directly on the surface. In some embodiments, the surface does not comprise an attractive moiety. In some embodiments, the plurality of vesicles are adsorbed unruptured to the surface. In some embodiments, causing the plurality of vesicles to rupture comprises contacting the plurality of vesicles with a plurality of lipid-rich vesicles, e.g., vesicles comprising POPC or POPC-PEG. In some embodiments, the surface comprises a coating or a chemical modification.

In some embodiments, the object is a glass microscope slide, a silicon wafer, a microfluidic channel, or a silica bead.

In some embodiments, the surface of the object is flat. In other embodiments, the surface of the object is round, curved, corrugated, or possesses another geometric shape or topography.

In some embodiments, the method further comprises separating the vesicles from one or more contaminants.

In some embodiments, the lipid bilayer comprises isolated plant plasma membranes derived from bubbles and blebs.

In some embodiments, the method does not comprise use of a detergent.

### BRIEF DESCRIPTION OF DRAWINGS

FIG. 1 is a schematic diagram showing a process for creating a supported lipid bilayer on a surface (protoplast-on-a-chip). A plant cell is subjected to enzymatic digestion to produce plasma membrane bubbles and free protoplasts. The free protoplasts are subjected to conditions resulting in the formation of plasma membrane blebs. The bubbles and blebs are contacted with a surface to produce a supported lipid bilayer on a surface (protoplast-on-a-chip).

FIG. 2A is a photomicrograph showing Arabidopsis thaliana cells expressing mCitrine-RCI2A.

FIG. 2B is a photograph showing protoplasts of Arabidopsis thaliana cells.

FIG. 2C is a photomicrograph showing Nicotiana benthamiana cells expressing PIP2A-mCherry.

FIG. 2D is a photograph showing protoplasts of Nicotiana benthamiana cells.

FIG. 2E is a photomicrograph showing Zea mays cells expressing PIN1-YFP.

FIG. 2F is a photograph showing protoplasts of Zea mays cells.

FIG. 3A is a schematic diagram depicting the generation of osmotically induced membrane bubbles from plant cells using enzymatic degradation of the plant cell wall by a cellulase enzyme. The upper left panel is an electron micrograph showing the dissolving cell wall. The upper right and lower panels illustrate a mechanism for bubble formation using the pressure gradient during digestion.

FIG. 3B is a schematic diagram depicting the generation of membrane blebs from plant cell protoplasts. This occurs by chemical induction using various chemical formulations.

FIG. 4A is a schematic diagram depicting a method for producing a supported plant plasma membrane, wherein membrane blebs or bubbles are adsorbed to and rupture on a glass surface coated with a polymer cushion designed to increase the interaction of the bubble or bleb with the surface by electrostatic forces.

FIG. 4B is a schematic diagram depicting a method for producing a supported plant plasma membrane, wherein membrane blebs or bubbles are adsorbed to a glass surface and rupture upon contacting with fusogenic vesicles comprising POPC and PEG5K.

FIG. 5 depicts imaging of fluorescently tagged membrane proteins in the plant bilayer.

FIG. 6 is a diagram showing potential applications for supported plasma membranes derived from plants.

FIG. 7A is a box plot depicting the zeta potential in millivolts (mv) of membrane bubbles and membrane blebs generated from Zea mays.

FIG. 7B is a box plot depicting the zeta potential in millivolts (mv) of membrane bubbles and membrane blebs generated from N. benthamiana.

FIG. 7C is a box plot depicting the zeta potential in millivolts (mv) of membrane bubbles and membrane blebs generated from Arabidopsis thaliana.

FIG. 7D is a graph showing particle size distribution in nanometers (nm) of membrane blebs produced from N. benthamiana as measured using a Malvern NanoSight.

FIG. 7E is a graph showing concentration in particles per mL of membrane blebs produced from N. benthamiana as measured using a Malvern NanoSight.

FIG. 7F is a graph showing zeta potential in mV of membrane blebs produced from N. benthamiana as measured using a Malvern ZetaSizer.

FIG. 8A is an electron micrograph showing a fully undigested N. benthamiana trichome.

FIG. 8B is an electron micrograph of N. benthamiana tissue showing two semi-digested parenchyma cell walls bordering each other with the lumen already released. An intact spherical chloroplast is shown next to the digested cell walls.

FIG. 8C is an electron micrograph of N. benthamiana tissue showing a detail of the secondary cell wall with clearly visible plasmodesmata.

FIG. 8D is an electron micrograph showing an intact chloroplast from N. benthamiana.

FIG. 8E is an electron micrograph showing an abundance of membrane bubbles from N. benthamiana spread over the chloroplast surface.

FIG. 9A is a set of photomicrographs depicting a fluorescence recovery after photobleaching (FRAP) experiment performed on a supported lipid bilayer from N. benthamiana. The panel labeled 0 s shows the fluorescent signal from the supported lipid bilayer before photobleaching. The panels labeled 1 s, 60 s, 600 s, and 1200 s show the fluorescent signal at 1, 60, 600, and 1200 seconds following photobleaching.

FIG. 9B is a graph showing intensity (counts per second (cps)) of fluorescence across a bleached area at multiple timepoints following photobleaching of a supported lipid bilayer from N. benthamiana.

FIG. 9C is a graph showing recovery of fluorescence after photobleaching (Recovery [%]) for a supported lipid bilayer from N. benthamiana. The experimental data is fitted to a Soumpasis equation.

FIG. 10 is a graph showing a Quartz Crystal Microbalance with Dissipation monitoring (QCM-D) profile of a Zea mays bleb ruptured with fusogenic POPC liposomes on a quartz sensor. F_1:3 shows the overtone three, showing the change in frequency. D_1:3 is the third overtone, representing the change in dissipation energy. The first 50 seconds depict equilibration of the sensor to the buffer. After 50 seconds, blebs are introduced, leading to an upshift in mass on the Quartz Crystal Microbalance (QCM) sensor, which is satisfied after about 200 seconds. Fusogenic liposomes are then introduced, leading to a mass upshift followed by the characteristic rupture curve when water is released from the vesicles and the bilayer is formed on the QCM chip.

FIG. 11A is a bar graph showing the number of single AtMate::YFP proteins having a given confinement radius (in μm) in a single particle tracking analysis in a supported plant plasma membrane. The percentage of tracked proteins that were confined is provided above the bar graph.

FIG. 11B is a plot showing the distribution of protein diffusivity from a single particle tracking analysis of AtMate::YFP proteins in a supported plant plasma membrane.

FIG. 11C is a bar graph showing the number of single AtMate::YFP proteins having a given diffusivity (in μm²/s) in a single particle tracking analysis in a supported plant plasma membrane.

FIG. 11D is a bar graph showing the distribution of protein diffusivity and mobility for AtMate::YFP proteins in a single particle tracking analysis in a supported plant plasma membrane. The average diffusivity of tracked proteins and the percent of tracked proteins that were mobile are provided above the bar graph.

FIG. 11E is a scatter plot showing the correlation between confinement of a protein and protein diffusivity. Parameter β describes the motion of a molecule as one of three states: β<0.4 is confined diffusion; 0.4≤β≤0.6 is quasi-free diffusion; and β>0.6 is convective diffusion.

FIG. 11F is a scatter plot showing the correlation between the length of a single protein's trajectory (in frames) and the diffusivity of the protein.

FIG. 11G is a plot showing the trajectories of each protein motion in the supported plant plasma membrane.

FIG. 11H is a photomicrograph showing fluorescence from each single fluorescent protein in the supported plant plasma membrane.

FIG. 12A is a photomicrograph (right panel) showing fluorescence of Octadecyl Rhodamine B Chloride (R18) on a glass slide at timepoint 0 and a schematic diagram (left panel) depicting the status of the experiment. R18-labeled Nicotiana benthamiana blebs (plant vesicles) are adsorbed to the surface, but have not ruptured.

FIG. 12B is a photomicrograph (right panel) showing fluorescence of R18 on a glass slide at timepoint 10 seconds and a schematic diagram (left panel) depicting the status of the experiment. Fusogenic vesicles comprising PEG5K and DOPC have been added to the surface comprising R18-labeled N. benthamiana blebs.

FIG. 12C is a photomicrograph (right panel) showing fluorescence of R18 on a glass slide at timepoint 20 seconds and a schematic diagram (left panel) depicting the status of the experiment. R18-labeled N. benthamiana blebs have ruptured in the presence of fusogenic vesicles comprising PEG5K and DOPC, and bilayer patches supported on the surface have formed.

FIG. 12D is a photomicrograph (right panel) showing fluorescence of R18 on a glass slide at timepoint 5 minutes and a schematic diagram (left panel) depicting the status of the experiment. R18-labeled N. benthamiana blebs have ruptured in the presence of fusogenic vesicles comprising PEG5K and DOPC, and a supported lipid bilayer on a surface (final plant membrane-on-a-chip) has formed.

FIG. 13A is a set of photomicrographs depicting a fluorescence recovery after photobleaching (FRAP) experiment performed on a supported lipid bilayer which includes N. benthamiana membrane bubbles. The panel labeled 0 shows the fluorescent signal from the supported lipid bilayer before photobleaching. The panels labeled 1, 2, and 3 show the fluorescent signal from the supported lipid bilayer at three timepoints following photobleaching, as annotated in FIG. 13B.

FIG. 13B is a graph showing recovery of fluorescence after photobleaching (Recovery [%]) for a supported lipid bilayer which includes N. benthamiana membrane bubbles. The experimental data is fitted to a Soumpasis equation.

FIG. 13C is a graph showing intensity (counts per second (cps)) of fluorescence across a bleached area before (0) and at three timepoints after (1, 2, and 3) photobleaching of a supported lipid bilayer which includes N. benthamiana membrane bubbles.

FIG. 14A is a graph showing recovery of fluorescence after photobleaching (Recovery [%]) for a supported lipid bilayer comprising Arabidopsis thaliana membrane bubbles. The experimental data is fitted to a Soumpasis equation.

FIG. 14B is a photomicrograph (left) showing the fluorescent signal from a photobleached area of a supported lipid bilayer comprising A. thaliana membrane bubbles and a graph (right) showing intensity (cps) of fluorescence across the photobleached area, as indicated by the line in the left panel, at multiple timepoints.

FIG. 14C is a set of photomicrographs showing the fluorescent signal from a supported lipid bilayer comprising A. thaliana membrane bubbles at 0 seconds, 30 seconds, and 2000 seconds after photobleaching.

FIG. 14D is a graph showing recovery of fluorescence after photobleaching (Recovery [%]) for a supported lipid bilayer comprising Arabidopsis thaliana membrane blebs. The experimental data is fitted to a Soumpasis equation.

FIG. 14E is a photomicrograph (left) showing the fluorescent signal from a photobleached area of a supported lipid bilayer comprising A. thaliana membrane blebs and a graph (right) showing intensity (cps) of fluorescence across the photobleached area, as indicated by the line in the left panel, at multiple timepoints.

FIG. 14F is a set of photomicrographs showing the fluorescent signal from a supported lipid bilayer comprising A. thaliana membrane blebs at 0 seconds, 30 seconds, and 2000 seconds after photobleaching.

FIG. 14G is a photomicrograph (left) showing the fluorescent signal from a photobleached area of a supported lipid bilayer comprising A. thaliana membrane blebs and a graph (right) showing intensity (cps) of fluorescence across the photobleached area, as indicated by the line in the left panel, at multiple timepoints.

FIG. 15A is a photomicrograph showing Arabidopsis thaliana cells expressing mCitrine-RCI2A.

FIG. 15B is a photomicrograph showing Arabidopsis thaliana cells expressing mCitrine-RCI2A.

FIG. 15C is a photomicrograph showing Arabidopsis thaliana cells without mCitrine-RCI2A as a control.

FIG. 15D is a Western blot showing the expression of mCitrine-RCI2A protein in transfected Arabidopsis thaliana cells.

FIG. 15E is a schematic diagram of the fusion protein mCitrine-RCI2A showing the fluorophore in the cytoplasm.

FIG. 15F is a schematic diagram of an assay for protein orientation of a supported lipid bilayer comprising mCitrine-RCI2A showing fluorophores oriented away from the surface being cleaved by Proteinase K.

FIG. 15G is a schematic diagram of an assay for protein orientation of a supported lipid bilayer comprising mCitrine-RCI2A showing fluorophores oriented toward the surface not being cleaved by Proteinase K.

FIG. 15H is a set of photomicrographs showing the fluorescent signal from a supported lipid bilayer assembled from A. thaliana membrane blebs comprising mCitrine-RCI2A (mCitrine blebs) at 0 minutes and 20 minutes after treatment with a protease.

FIG. 15I is a set of photomicrographs showing the fluorescent signal from a supported lipid bilayer assembled from A. thaliana membrane blebs not comprising a fluorescently labeled protein (blank blebs) at 0 minutes and 20 minutes after treatment with a protease.

FIG. 16 is a schematic diagram showing a protocol for single-molecule tracking in a supported lipid bilayer. The left panel shows a cell expressing MCitrine-RCI2A. The center panel shows a supported lipid bilayer comprising mCitrine-RCI2A in a microfluidic channel, wherein the mCitrine fluorophore is excited and emits a fluorescent signal that is captured by an objective lens of a fluorescence microscope. The right panel shows puncta of fluorescence in the supported lipid bilayer that may be tracked.

FIG. 17A is a photomicrograph showing intact cell blebs expressing PIP2A-mCherry adsorbed on glass slides. The fluorescent signal is from PIP2A-mCherry.

FIG. 17B is a photomicrograph showing PIP2A-mCherry incorporation after formation of a supported plant lipid bilayer from plant plasma membrane blebs. The fluorescent signal is from PIP2A-mCherry.

FIG. 18A is a plot showing trajectories of single protein diffusion in a planar supported plant lipid bilayer and a schematic diagram showing the PIP2A-mCherry protein embedded in a plasma membrane.

FIG. 18B is a bar graph showing the diffusivity of single PIP2A-mCherry tracked particles.

FIG. 18C is a scatter plot showing the diffusivity of single PIP2A-mCherry tracked particles.

### DETAILED DESCRIPTION OF THE INVENTION

### I. Definitions

As used herein, the term “plant plasma membrane” refers to a lipid bilayer structure included in or derived from a plant cell. The plant plasma membrane may consist of or be derived from any membrane of a plant cell, e.g., a source plasma membrane, e.g., a cell membrane (e.g., a cytoplasmic membrane; plasmalemma), a vacuole membrane (e.g., a tonoplast), an organelle membrane (e.g., a chloroplast membrane (e.g., a thylakoid membrane or an inner or outer membrane of the chloroplast), a mitochondrial membrane (e.g., a crista membrane or an inner or outer membrane of the chloroplast), a nuclear membrane (e.g., a nuclear envelope), a Golgi membrane, an endoplasmic reticulum membrane, or a vesicle membrane. The plant plasma membrane may be planar in form, or may have a form other than planar (e.g., a closed membrane structure, e.g., a vesicle, e.g., a bubble or a bleb). The plant plasma membrane may contain one or more plant proteins, e.g., plant proteins embedded in or associated with the membrane. In some instances, a plant plasma membrane is an intact plasma membrane of a plant cell. In other instances, the plant plasma membrane is assembled from (e.g., self-assembles from) a plurality of moieties (e.g., vesicles, e.g., bubbles and/or blebs) comprising plant plasma membranes. The moieties may comprise plasma membranes from one or a plurality of source plasma membranes and from one or a plurality of plant cells. In some examples, the plant plasma membrane is not associated with a plant cell, e.g., is not associated with a plant cell wall. Plant plasma membranes may be derived from any suitable plant, e.g., any angiosperm (e.g., tomato, maize, or Arabidopsis), gymnosperm, fern, moss, hornwort, liverwort, alga, or cyanobacterium.

As used herein, the term “bubble” refers to a vesicle comprising a plant plasma membrane that has been produced using a method including treating a plant cell to disrupt the cell wall, e.g., treating a plant cell with a cellulase or a pectinase. In some examples, bubbles are produced by osmotic pressure.

As used herein, the term “bleb” refers to a vesicle comprising a plant plasma membrane that has been produced using a method that includes treating a plant cell protoplast to induce blebbing, e.g., treating the plant cell protoplast with a blebbing buffer, e.g., treating the plant cell protoplast with a buffer comprising formaldehyde or DTT.

The plant plasma membrane may be supported on a surface. As used herein, the terms “supported lipid bilayer”, “supported plant plasma membrane”, “supported on a surface” or “supported” describes a lipid bilayer that is associated with and conforms to a surface of an object, and that is not associated with a plant cell. As used herein, the term “surface” refers to a surface of an object with which a plant plasma membrane may associate. The surface may be flat, round (e.g., a surface of a round object), curved, or corrugated, or may possess any other geometric shape or topography. As used herein, “adsorbed to the surface” describes a lipid bilayer that is associated with, but not covalently attached to, a surface or a coating on a surface. For example, the lipid bilayer may have ionic interactions with the surface. “Adhering to the surface” or “adheres to the surface” describes a lipid bilayer that is covalently attached to a surface or a coating thereof. Plant plasma membranes that are supported on a surface may be adsorbed to the surface or a coating thereof, may adhere to (e.g., be covalently linked to) the surface or a coating thereof, or may be otherwise associated with the surface or coating thereof.

### II. Supported Plant Plasma Membranes

In general, the disclosure features compositions including a plant plasma membrane supported in the surface of an object, wherein the plant plasma membrane is not associated with a plant cell, and methods for manufacturing said compositions.

Plant plasma membranes are comprised of various amphipathic lipid molecules and proteins, e.g., proteins crucial for cell functions such as signal recognition and transduction into intracellular responses as well as environmental responses and developmental signaling. Proteins are regulated by a complex cascade of protein-protein and lipid-protein interactions in various ways. The cell plasma membrane encapsulates the cell cytosol and organelles and plays a crucial role in all interactions with the external environment. The plant PM is clearly the most manifold membrane of a plant cell. Its composition of lipids and associate proteins is dynamic and varies with cell type, developmental stage and environmental conditions to allow the formation of a selectively permeable barrier for the specific uptake of required macromolecules and solutes and the blocking of others. Although they are sessile organisms, plants need to adapt constantly to a dynamic environment in order to supply their needs and fend off pathogens.

To understand how membrane proteins (e.g., signaling cascades of membrane proteins) function in biological membranes, in vivo (cell-based) assays are predominantly used. The clear advantages of these cell-based assays is the preservation of the native environment (e.g. presence of the cell wall); however, this advantage of working with total cells is also its biggest disadvantage. The isolation and measurement of individual factors in a system of still-unknown complexity and dynamism makes such assays inconvenient and error-prone. Traditional characterization techniques for lipid-protein interaction often use detergents to extract membrane proteins; however, processing with detergents can affect protein function as well as the native lipid-protein association. Such studies also experience difficulties with isolating single events from the surrounding structures, such as the cell wall or the vacuole.

Previous methods for making isolated lipid bilayers have used commercially available lipids and reconstituted species of interest. In contrast to these previous methods, the methods and compositions described herein produce plant plasma membranes having native protein and lipid orientation and dynamics. These methods and compositions bridge in vivo and in vitro assays, are robust, and are compatible with a wide variety of surface characterization tools such as total internal reflection microscopy (TIRFM), atomic force microscopy (AFM), quartz crystal microbalance (QCM), and surface plasmon resonance (SPR), making the methods and compositions a valuable tool for unveiling new insights into plant membrane protein function, transport phenomena, and host-pathogen interactions.

Features that distinguish this Invention are: the methods and compositions preserve the native membrane constituents found in the plant cell (lipids proteins, other native species); the methods and compositions do not require purifying any species out of the membrane, namely proteins, which can easily denature when reconstituted back into membranes; the methods and compositions use a self-assembly process to create the membrane on a chip; the methods and compositions preserve lipid and protein mobility in the two-dimensional plane of the membrane on the chip; the methods and compositions are compatible with molecular biology tools that allow genetic manipulation of the membrane constituents, such as introduction of mutant proteins, deletion of proteins, addition of exogenous proteins, and so on; and it is possible to study plant membrane fusion with the methods and compositions following protocols developed for viruses fusing with planar mammalian membranes.

A. Plant Plasma Membranes

In one aspect, the disclosure features a composition comprising a plant plasma membrane supported on a surface of an object, wherein the plant plasma membrane is not associated with a plant cell. In some examples, the plant plasma membrane is a lipid bilayer. The plant plasma membrane may alternatively be a unilamellar (e.g., consisting of only one a single layer of lipids) or multilamellar (e.g., including multiple lipid bilayers.

i. Source Plasma Membranes

The plant plasma membrane supported on the surface may be partially or entirely derived from (e.g., may consist of) any plasma membrane of a plant cell (e.g., a source plasma membrane). In some examples, the plant plasma membrane supported on the surface is partially or entirely derived from a single type of plant plasma membrane. For example, the supported plant plasma membrane may be derived from the cell membrane (cytoplasmic membrane; plasmalemma) of one or more cells. In another example, the supported plant plasma membrane is derived from a chloroplast membrane (e.g., a thylakoid membrane). In other examples, the supported plant plasma membrane may be derived from a vacuole membrane (e.g., a tonoplast), a mitochondrial membrane (e.g., a crista membrane or an inner or outer membrane of the chloroplast), a nuclear membrane (e.g., a nuclear envelope), a Golgi membrane, an endoplasmic reticulum membrane, or a vesicle membrane. Alternatively, the supported plant plasma membrane may be derived from more than one plasma membrane of a plant cell. Methods for separating plant plasma membranes from a source cell of a source plant or plant part are described below. In brief, the supported plant plasma membrane may be produced by causing a plurality of vesicles (e.g., bubbles or blebs, as described herein) comprising a plant plasma membrane to rupture on a surface of an object and to assemble into a continuous lipid bilayer on said surface.

The supported plant plasma membrane may have a protein orientation, protein activity, lipid orientation, or protein or lipid mobility that is comparable to or identical to the protein orientation, protein activity, lipid orientation, or protein or lipid mobility of proteins or lipids of the source plasma membrane in its native context in the source cell. For example, at least 10%, 15%, 20%, 25%, 30%, 35%, 40%, 45%, 50%, 55%, 60%, 65%, 70%, 75%, 80%, 85%, 90%, 95%, 99%, or 100% of all proteins or of a given protein of interest may have the same orientation in the supported plant plasma membrane as in the source plasma membrane. At least 10%, 15%, 20%, 25%, 30%, 35%, 40%, 45%, 50%, 55%, 60%, 65%, 70%, 75%, 80%, 85%, 90%, 95%, 99%, or 100% of all proteins or of a given protein of interest may have the same level of activity (e.g., catalytic activity, signaling activity, or transporter activity) in the supported plant plasma membrane as in the source plasma membrane. Protein or lipid mobility may be reduced or increased by less than 1%, less than 2%, less than 3%, less than 4%, less than 5%, less than 10%, less than 15%, less than 20%, less than 25%, less than 30%, less than 35%, less than 40%, less than 45%, less than 50%, less than 55%, less than 60%, less than 65%, less than 70%, or less than 75% relative to the source plasma membrane, e.g., as measured by percent confinement, mobility, or diffusivity. The supported plant plasma membrane may completely or partially have the heterogeneity of the source plant plasma membrane, e.g., may contain all or a subset of the species (e.g., proteins, lipids, and other membrane components) present in the source plant plasma membrane. For example, the supported plant plasma membrane may contain at least 10%, 15%, 20%, 25%, 30%, 35%, 40%, 45%, 50%, 55%, 60%, 65%, 70%, 75%, 80%, 85%, 90%, 95%, 99%, or 100% of the species present in the source plasma membrane. Other features from the source plasma membrane, e.g., lipid domains, may also be included in the supported plant plasma membrane,

ii. Plants and plant parts

The plant plasma membrane may be derived from any plant or part thereof. For example, the plant plasma membrane may be derived from an angiosperm, a gymnosperm, a fern, a moss, a hornwort, a liverwort, an alga, or a cyanobacterium. Plants from which the plant plasma membrane can be derived include, but are not limited to acacia, alfalfa, apple, apricot, Arabidopsis, artichoke, ash tree, asparagus, avocado, banana, barley, beans, beet, birch, beech, blackberry, blueberry, Brachypodium, broccoli, brussels sprouts, cabbage, canola, cantaloupe, carrot, cassava, cauliflower, cedar, a cereal, celery, chestnut, cherry, Chinese cabbage, Chlamydomonas, citrus, clementine, clover, coffee, corn (maize), cotton, cowpea, cucumber, cypress, eggplant, elm, endive, eucalyptus, fava beans, fennel, figs, fir, geranium, grape, grapefruit, groundnuts, ground cherry, gum hemlock, hickory, kale, kiwifruit, kohlrabi, larch, lettuce, leek, Lemna, lemon, lime, locust, Lotus, pine, maidenhair, maize, mango, maple, Marchantia, Medicago, melon, millet, Mimulus, mushroom, mustard, nuts, oak, oats, okra, onion, orange, an ornamental plant or flower or tree, papaya, palm, parsley, parsnip, pea, peach, peanut, pear, peat, pepper, persimmon, Physcomitrella, pigeon pea, pine, pineapple, plantain, plum, pomegranate, Populus, potato, pumpkin, radicchio, radish, rapeseed, raspberry, rice, rye, sorghum, sallow, Selaginella, soybean, spinach, spruce, squash, strawberry, sugarbeet, sugarcane, sunflower, sweet potato, sweet corn, tangerine, tea, tobacco, tomato, trees, triticale, turf grasses, turnips, a vine, walnut, watercress, watermelon, wheat, yam, yew, and zucchini. In some examples, the plant plasma membrane is derived from Arabidopsis (e.g., Arabidopsis thaliana), tobacco (e.g., Nicotiana benthamiana), or maize (e.g., Zea mays).

The supported plant plasma membrane may be derived from any plant part or plant tissue, including a leaf or a portion of a leaf, a stem, or a root of a plant. Other suitable plant parts and plant tissues include, but are not limited to shoots, tubers, stolons, rhizomes, stems, pollen, callus tissue, cell culture, somatic embryos, embryos, flowers, and fruit. The plant part may be processed (e.g., chopped or blended).

iii. Modified Plant Plasma Membranes

The supported plant plasma membrane may be modified. In some examples, the modification is the inclusion of one or more exogenous proteins, e.g., an exogenous protein embedded in or associated with the supported plant plasma membrane. The exogenous protein may optionally include a tag or marker, e.g., may be a fusion protein including a fluorescent moiety. In other examples, the modification is the exclusion of one or more proteins that are native to the source plasma membrane in a wild-type cell comprising the source plasma membrane. The modification may be the addition or exclusion of one or more other membrane components, e.g., an amphiphilic molecule, a lipid, a glycolipid, a cholesterol or a derivative thereof, a polymer species, a hormone, or a synthetic membrane component. The modification may be the addition of a dye or stain, e.g., a lipophilic dye or stain. In some instances, the modification is made to the source plasma membrane (e.g., source cell membrane or source chloroplast membrane) in the context of the source cell. The source cell may be stably or transiently genetically modified, e.g., genetically modified to express an exogenous protein of interest or to produce an increased or decreased (e.g., abrogated) level of a biomolecule (e.g., protein) native to the source cell. Methods for the genetic modification of plant cells are known in the art. In other examples, the plant cell may be exposed to an environmental condition (including, but not limited to light, temperature, drought stress, salt stress, or exposure to a chemical agent) that causes a modification of the source plasma membrane of the plant cell. Alternatively, the plant plasma membrane may be modified after the plant plasma membrane is separated from the source cell. For example, a bubble or bleb comprising the plant plasma membrane may be modified. In another example, the plant plasma membrane is modified during or after the assembly of the plant plasma membrane on the surface, as is described below. In brief, the plant plasma membrane may be altered during or after assembly by providing vesicles (e.g., synthetic vesicles) or other membrane components that are assimilated into the lipid bilayer.

iv. Surfaces and Modifications Thereof

The supported plant plasma membrane may be supported on any suitable surface of any suitable object. The surface may have any shape, geometry, or topography. In some examples, the surface is flat. In other examples, the surface is round (e.g., is the surface of a round object, e.g., a bead), curved, or textured (e.g., corrugated). The supported plant plasma membrane may be associated with and may conform to the surface of the object, and may be non-covalently attached to (adsorbed to) the surface or a coating or chemical modification on the surface. Alternatively, the supported plant plasma membrane may adhere to (e.g., be covalently attached to) the surface or a coating or chemical modification thereof. Methods for promoting the association of a plant plasma membrane with a surface are provided herein.

The surface may be composed of, e.g., glass or silica. Alternatively, the surface may be composed of any material that partially or completely promotes the rupture of vesicles (e.g., bubbles or blebs) comprising plant plasma membranes onto the surface. Such surfaces include, but are not limited to polyelectrolyte surfaces, PMETAC (Poly[(2-(Methacryloyloxy)Ethyl) Trimethylammonium Chloride]) surfaces, PEDOT:PSS (poly(3,4-ethylenedioxythiophene) polystyrene sulfonate) surfaces, PEG (polyethylene glycol) surfaces, PLL (poly(L-lysine)) surfaces, or cellulose surfaces.

The surface may comprise a coating, e.g., a polymer coating, e.g., a polyelectrolyte, PMETAC, PEDOT:PSS, PEG, PLL, or cellulose coating. Said coating may promote the rupture of vesicles (e.g., bubbles or blebs) comprising plant plasma membranes onto the surface. In some examples, the coating is PMETAC. Surfaces or coatings thereof that may be used to promote the rupture of vesicles include hydrophilic moieties, e.g., moieties that provide an environment similar to that of a native plant plasma membrane. Such moieties may passivate and cushion the bilayer so as to limit the denaturing of proteins on the surface, or may promote the overall interaction between the membrane and the surface by increasing attractive forces, for example electrostatic forces or van der Waals.

The surface may be treated, e.g., chemically modified, to promote the rupture of vesicles (e.g., bubbles or blebs) comprising plant plasma membranes onto the surface. Chemical modifications to the surface may include, e.g., silanes, amine groups, and coupling groups. Other chemical modifications to the surface may include the addition of one or both members of an affinity binding pair, e.g., one or both of biotin and avidin, wherein the affinity binding pair is biotin and avidin; one or both of biotin and streptavidin, wherein the affinity binding pair is biotin and streptavidin; one or both of an antibody or antibody fragment (e.g., an IgG antibody or antibody fragment) and an antigen, wherein the affinity binding pair is the antibody and the antigen; or one or both strands of a complementary DNA, wherein the affinity binding pair is the duplex of the two strands of complementary DNA (DNA complementary tethering). In some examples, the vesicles (e.g., bubbles or blebs) may comprise a member of the affinity binding pair, e.g., may comprise a member of the affinity binding pair the binding partner of which is comprised by the surface. The vesicles (e.g., bubbles or blebs) may be modified to comprise a member of the affinity binding pair. In some examples, the surface is hydrophilic, or comprises a coating or chemical modification that causes the surface to be hydrophilic.

In some examples, the object is a glass microscope slide and the surface is a surface thereof; the object is a silicon wafer and the surface is a surface thereof; the object is a microfluidic device and the surface is a surface thereof (e.g., a surface of a microfluidic channel); or the object is a silica bead and the surface is the surface or a portion of the surface thereof.

B. Methods

In a second aspect, the disclosure features a method for manufacturing a plant plasma membrane (e.g., a lipid bilayer) supported on a surface of an object, the method comprising (a) providing a plurality of vesicles, wherein each vesicle comprises an isolated plant plasma membrane; and (b) contacting the plurality of vesicles with a surface of an object, wherein the isolated plant plasma membranes assemble into a continuous lipid bilayer, thereby forming the plant plasma membrane supported on the surface of the object. In some examples, the method does not comprise use of a detergent.

i. Production of Bubbles

In some embodiments, the plurality of vesicles comprising a plant plasma membrane is a plurality of bubbles, and providing the plurality of vesicles comprises treating a plant or plant part to produce a plurality of bubbles.

As used herein, the term “bubble” describes a vesicle comprising a plant plasma membrane that has been produced by a process including disrupting a portion of a cell wall of a plant cell such that fragments of a cell membrane (vesicles or “bubbles”) are extruded at the site of the disruption and are separated from the source plant plasma membrane, e.g., by a pressure differential of the cell cytoplasm relative to the extracellular environment (e.g., a solution in which the plant or plant part is placed).

The treating of the plant or plant part may comprise administering one or more agents that disrupt a cell wall of the plant or plant part. The agent may be, e.g., a cellulase, or a pectinase. In some aspects, the agent comprises a cellulase and a pectinase. The agent or agents may be administered in an amount and for a time sufficient to cause degradation of the cell wall to produce bubbles.

ii. Production of Blebs

In some embodiments, the plurality of vesicles comprising a plant plasma membrane is a plurality of bubbles, and providing the plurality of vesicles comprises (a) providing a plant protoplast and

### (b) Treating the Plant Protoplast to Produce a Plurality of Blebs.

As used herein, the term “bleb” describes a vesicle comprising a plant plasma membrane that has been produced by a process including treating a plant protoplast to produce smaller fragments comprising a plant plasma membrane (vesicles, “blebs”, or proteoliposomes) that are separated from the source plant plasma membrane.

Providing the plant protoplast may comprise producing the plant protoplast from a plant cell, e.g., by treating the plant cell with one or more agents that disrupt the cell wall. The agent may be, e.g., a cellulase, a pectinase, or a cellulase and a pectinase. The agent or agents may be administered in an amount and for a time sufficient to cause degradation of the cell wall to produce a protoplast. Protoplasts may be produced from any of the plants or plant parts described herein.

Methods for producing blebs from protoplasts are known in the art. The two most commonly used methods are serum starving and chemical induction of blebbing. Serum starving is characterized by the removal of all serum, an essential component of a mammalian cell culture, which is followed by a cascade of stress responses by the mammalian cell. One of these responses is the secretion of exosomes (blebs). Serum starving may also be used to induce blebbing of plant protoplasts. Chemical induction of blebbing comprises administering a chemical substance that induces blebbing of the protoplasts. Methods for making protoplast blebs are known in the art, and any suitable agent, method, or condition causing the production of membrane blebs may be used. Formaldehyde (FA) and dithiothreitol (DTT) are commonly used to chemically induce blebbing. DTT reduces disulfide bonds and cysteines, which is affects lipid phase partitioning. FA facilitates local anomalies by functioning as a protein cross-linking agent. Alternatively, blebs may be produced by inducing osmotic stress in the protoplasts, e.g., by contacting the protoplasts with a hypotonic or hypertonic solution.

In some embodiments, the method comprises providing a plurality of vesicles, wherein the plurality of vesicles comprises both bubbles and blebs. In this method, the providing comprises (a) treating a plant or plant part to produce a plurality of bubbles; (b) treating the plant or plant part of step (a) to produce a plant protoplast; and (c) treating the plant protoplast of step (b) to produce a plurality of blebs. The treating of step (a) may comprise, e.g., administering one or more agents that disrupt a cell wall of the plant or plant part, e.g., a cellulase, a pectinase, or a cellulase and a pectinase. The treating of step (b) may be the treating of step (a), e.g., may comprising continuing the treatment of step (a) such that bubbles are produced at an earlier time point in the treatment, when the cell wall has been partially degraded, and protoplasts are produced at a later time point in the treatment, when the cell wall has been mostly or entirely degraded. In another example, the treating of step (b) may be a treatment other than or in addition to the treatment of step (a), e.g., may comprise further chemical and/or physical processing. Bubbles and/or blebs may be collected from the solution (e.g., purified or separated from cell wall fragments, plant parts, or other unwanted components of the solution) at one or more than one stage in the production process.

The plant, plant part, or plant cell may be any plant, plant part, or plant cell described herein, e.g., as described in Section IIA(ii). In some examples, the plant, plant part, or plant protoplast is a maize plant, plant part, or protoplast, an Arabidopsis plant, plant part, or protoplast, or a tobacco plant, plant part, or protoplast. In some examples, the plant part is a leaf, stem, or root of a plant. The source plasma membrane may be any source plasma membrane described herein, e.g., as described in Section IIa(i). In some examples, the source plasma membrane is a cell plasma membrane (plasmalemma). In other examples, the plant part is a chloroplast and the source plasma membrane is a chloroplast membrane (e.g., a thylakoid membrane). In some examples, the plant plasma membrane is modified, e.g., as described in Section IIA(iii) herein.

iii. Production of a Supported Plasma Membrane on a Surface

The methods described herein include contacting a plurality of vesicles with a surface of an object, wherein the isolated plant plasma membranes assemble into a continuous lipid bilayer, thereby forming the plant plasma membrane supported on the surface of the object. The surface may be any surface described herein, e.g., as described in Section IIa(iv). In some examples, the contacting comprises causing the plurality of vesicles to rupture on a surface, wherein the ruptured vesicles create a continuous lipid bilayer.

Any suitable method may be used to cause or promote the association of the vesicles with the surface and the rupture of the vesicles onto the surface. In some instances, the method comprises providing an attractive (e.g., hydrophilic) surface on which the vesicles rupture. In other instances, the method comprises adsorbing unruptured vesicles (e.g., bubbles or blebs) to a surface and contacting the unruptured vesicles with fusogenic vesicles, thereby causing the vesicles comprising the plant plasma membrane to rupture on the surface.

Methods Comprising Attractive Surfaces

In some aspects, causing the plurality of vesicles to rupture comprises providing a surface comprising one or more attractive moieties, wherein the one or more attractive moieties partially or completely promote the rupture of the vesicles onto the surface. The attractive moiety may be a surface or a surface coating, e.g., a polymer surface or surface coating, e.g., a polyelectrolyte surface or coating, a PMETAC surface or coating, a PEDOT:PSS surface or coating, a PEG surface or coating, a PLL surface or coating, or a cellulose surface or coating. In some examples, the surface is a PMETAC surface. The attractive moiety may be a chemical modification of the surface, e.g., a chemical modification comprising a silane, an amine group, or a coupling group. In some examples, the surface is hydrophilic, or comprises a coating or chemical modification that causes the surface to be hydrophilic.

Methods Comprising Fusogenic Vesicles

In some embodiments, the plurality of vesicles (e.g., bubbles or blebs) comprising plant plasma membranes is adsorbed to the surface, but the plurality of vesicles does not initially rupture on the surface. The surface may not comprise an attractive moiety. An additional step may be taken to cause the vesicles to rupture on the surface. In some examples, causing the plurality of vesicles to rupture comprises contacting the plurality of vesicles (e.g., bubbles or blebs) with a plurality of fusogenic vesicles, e.g., lipid-rich vesicles. The lipid-rich vesicles may comprise any phospholipid that spontaneously fuses onto glass under appropriate conditions, e.g., POPC, POPC-PEG, or DOPC. The supported plant plasma membrane may be composed of between 0% and 99% material from ruptured fusogenic vesicles, e.g., 1%, 5%, 10%, 15%, 20%, 25%, 30%, 35%, 40%, 45%, 50%, 55%, 60%, 65%, 70%, 75%, 80%, 85%, 90%, 95%, or 99% material from ruptured fusogenic vesicles. In some instances, the fusogenic vesicles (e.g., POPC-PEG vesicles) further modify the plant plasma membrane or the composition comprising the plant plasma membrane and the surface, e.g., create a larger space between the plant plasma membrane and the surface, thus increasing protein mobility. In some examples, the method includes providing a surface having an attractive moiety and contacting the surface and the plurality of vesicles with a plurality of fusogenic vesicles.

### III. Applications

The methods and compositions described herein may be used, e.g., for various basic and applied science studies. For example, the supported plant plasma membranes described herein may be used for various basic science studies, e.g., studying the interactions between and activity and function of plant proteins and lipids. This may be conducted in various manners, including single protein tracking microscopy comprising imaging of a supported plant plasma membrane described herein. In another example, the supported plant plasma membrane is used for the study of ion channel function and dependence on environmental conditions to understand, for example, water transport or hormone transport in plants, or other biological functions. Such studies could lead to more robust plants and identify genetic factors that lead to resilient plants. Additionally, the supported plant plasma membranes may be used in basic biophysical studies of membrane fluidity, stability, heterogeneity, and other mechanical properties as they vary with growth conditions of the plant, genetic changes, pathogen interactions, exposure to chemicals like herbicides, fertilizers, etc.

Further, the methods and compositions described herein may be used for various applied studies, e.g., the study of pathogen interaction with the membrane (binding) and processes that lead to cell infection and disease spread; studies of processes like the fusion of two plant membranes in the process of protoplast fusion; a screening platform for aspects of plant membrane function and stability; or to create a ‘leaf-on-a-chip’ as a sensor or solar cell using plant materials and appropriate surface and electronics integration, for example with the organic electrochemical transistor device, but could be integrated with many other electrical systems or sensing transducers. This platform can be merged with existing approaches for cell-free biomanufacturing of therapeutic proteins and other biomaterials by leveraging the plant enzymes that produce these species on our chip platform.

The planar geometry of some embodiments the supported plant plasma membrane makes the platform compatible with various microscopy tools, surface analysis tools, and microfluidic channels for high throughput studies.

### IV. Examples

### Example 1. Production and Characterization of Supported Plant Plasma Membranes

In this example, supported plant lipid bilayers from Arabidopsis, maize, and tobacco were produced and characterized. Two different methods were used to generate vesicles and blebs, which were examined using several approaches to investigate the impact of the method of formation on the structure and functionality of the vesicles.

Techniques were tested on a monocotyledon (Zea mays) and dicotyledonous plant species (Nicotiana benthamiana and Arabidopsis thaliana) in parallel, and the resulting blebs were characterized in size, charge, and concentration. Supported lipid bilayers (SLBs) formed with each fraction were characterized for diffusivity, confinement, protein orientation, protein mobility, and activity. The lipid composition and protein content of the plasma membrane influences the lipid-protein and protein-protein associations, membrane-bound enzyme activities, as well as permeability of the membrane, which makes it an interesting target to study.

This Example demonstrates that planar plant cell membrane bilayers using cell blebs or bubbles as an intermediate can be made from several plant varieties as shown. Because of their planar geometry, these bilayers are compatible with a vast array of surface characterization tools. A key advantage of bubble or bleb bilayers is the ability to create them without the use of detergent or reconstitution, while maintaining the richness in composition of the cell membrane itself and the native protein orientation and activity. This work represents the first ever approach to characterized cell membrane properties from plant (N. tabacum, Z. mays, and Arabidopsis) cells by comparing two different formation conditions. We show that both methods were suitable to form a 100% supported lipid bilayer with a high percentage of mobile proteins and a very low confinement for PIN1, AtMate, PIP2A, and RCI2A.

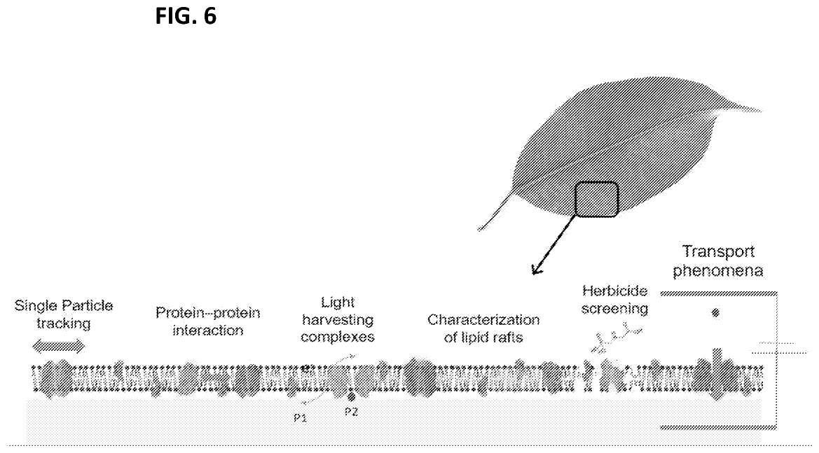

This Example shows the formation of a molecularly complete planar supported plant cell membrane derived from intact protoplasts with maximally functional proteins. We describe two economic and reliable ways to derive microvesicles (bubbles and blebs) of different qualities from the plant cells and to rupture them to produce a supported plant lipid bilayer. We confirmed that both methods preserve the protein orientation, mobility, and activity of proteins in the intact plasma membrane in the full plant cell. We observed protein confinement in the SLBs, and partially resolved this issue by applying PEGylated POPC lipids as a cushion, adding 6 nm of extra space for the cytosolic part of the proteins. This novel platform is a powerful tool for the plant science community for various in vitro studies of the cell surface architecture and transport phenomena, as well as quantitatively measuring virus binding and cell entry. With the extensive characterization presented here, we open up a number of possibilities for the use of the disclosed platform, from tissue engineering to a phenotypical selection tool for plant breeding, herbicide screening to advanced studies of virus binding and investigation of transport phenomena across the most complex membrane of a plant cell (FIG. 6).

a) Marker Proteins and Plant Lines

Candidate proteins to test this platform were chosen based on structure, molecular weight and number of transmembrane loops. We selected four transmembrane proteins: PIN1, PIP2A, AtMate, and RCI2A (AT3G05880), which are involved in variety of biological process such as auxin signaling. Each protein comprised a fluorescent marker, as described below. The native lipid-to-protein mass ratio for the plant PM approximately 1:1; however, taking into account that the molar mass of a lipid molecule is far below the average protein molecular mass, the lipid-to-protein molar ratio in the plant PM ranges around 75:1.

Stably transformed PIN1-YFP Z. mays lines were obtained from the Jander research group at the Boyce Thompson Institute (BTI). PIN1 is a membrane-bound auxin carrier protein involved in auxin transport and gravity response of the plant, as well as upright growth. In the PIN1-YFP construct, a yellow fluorescent protein is fused to the C-terminus.

AtMATE::mCherry, AtMATE::YFP, and PIP2A::mCherry constructs were used for transient expression in N. benthamiana after syringe infiltration in 4 week old plants. These transiently transformed plants were used for protoplast isolation and production of blebs and bubbles. Plants were cultivated at 23° C., 50% rel. humidity and 12 h light cycles pre and post infiltration.

A stably transformed mCitrine-RCI2A Arabidopsis thaliana line was obtained, as described in Robinson et al. (Plant Cell, 30: 2308-2329, 2018).

b) FRAP, QCM-D, and TIRFM

Supported plant plasma membranes were examined using the following approaches:

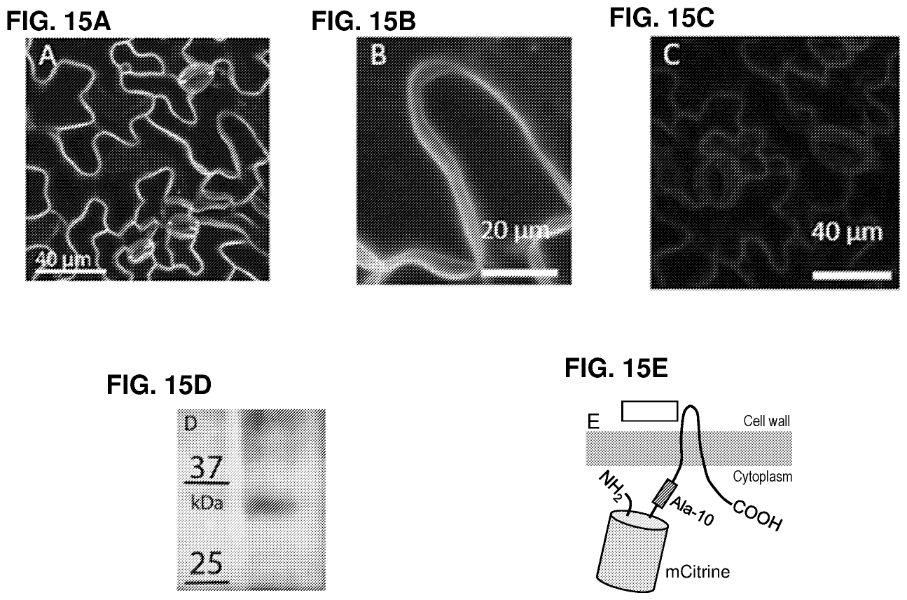

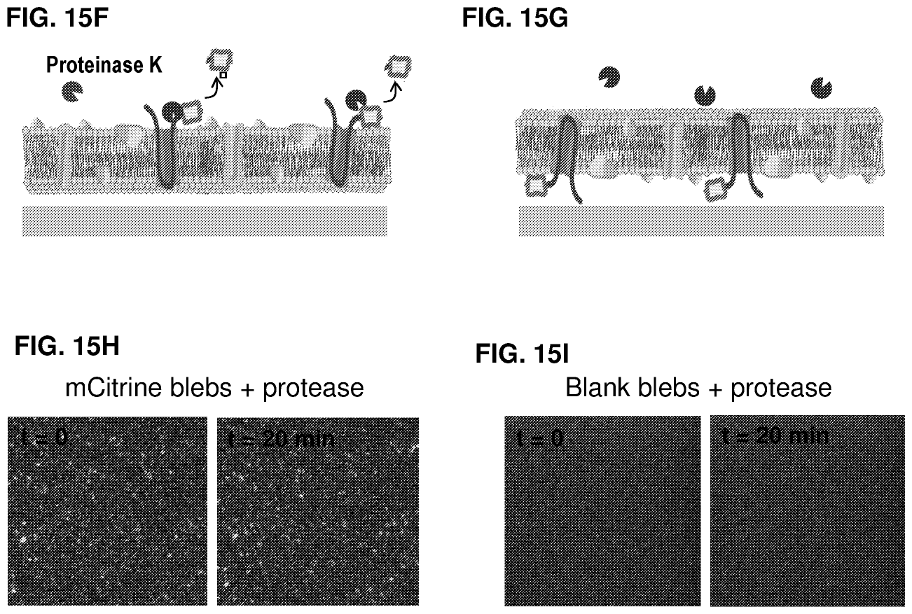

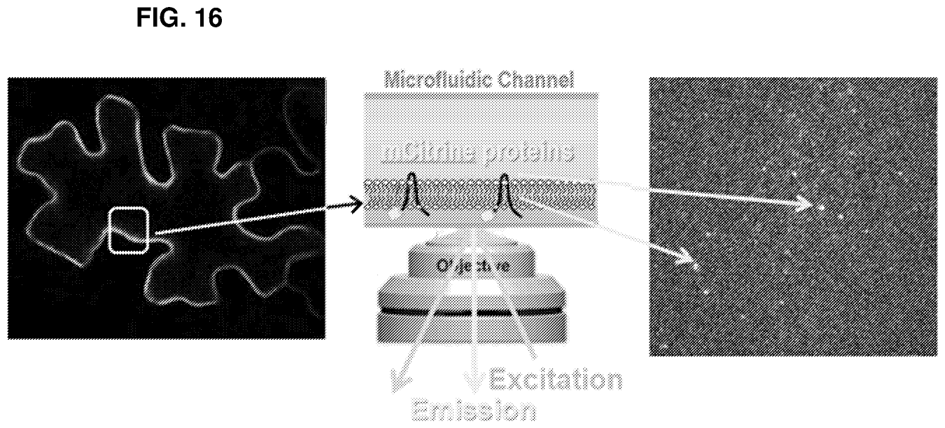

fluorescent recovery after photo bleaching (FRAP); quartz crystal micro balance with dissipation (QCM-D); and total internal reflection microscopy (TIRFM). Native plant tissue labeled with Rhodamine dye (R18) was used for the FRAP experiments, and unlabeled vesicles were used for the QCM-D experiments. To assess protein mobility transiently expressed candidate proteins (Pin1, AtMate and PIP2A) were expressed to study protein mobility via single particle tracking (SPT) using TIRFM. For the SPT, the single fluorescently labeled protein is tracked in a TIRF field of around 94 nm thick, ensuring surface specificity to determine the protein mobility and diffusivity in the bilayer for each method. To confirm the original protein orientation after blebbing and rupture, a protease cleavage assay was used to determine the side of the attached fluorophore (FIGS. 15F-15I).

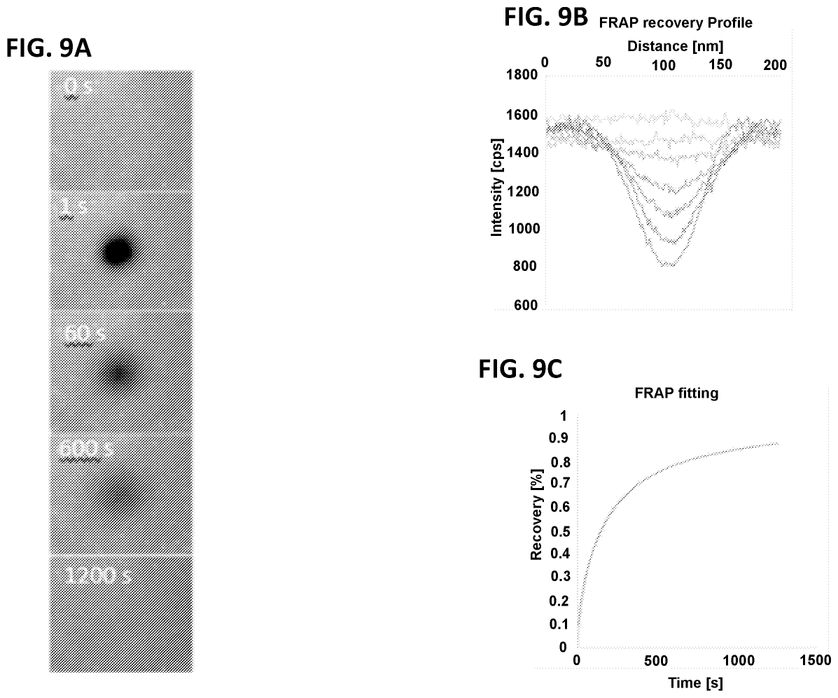

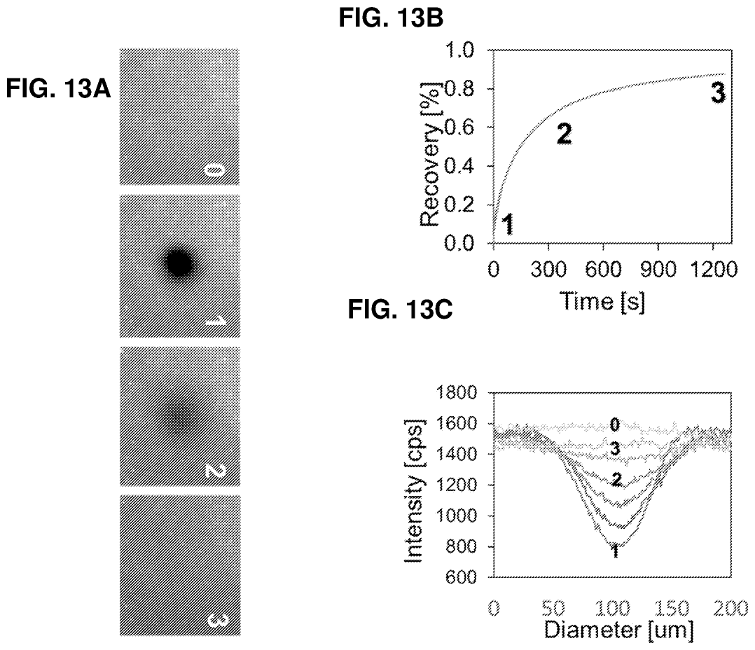

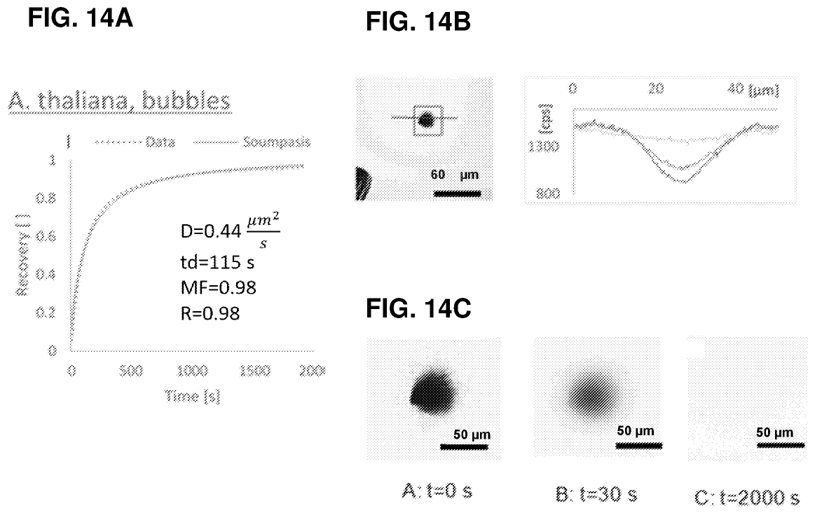

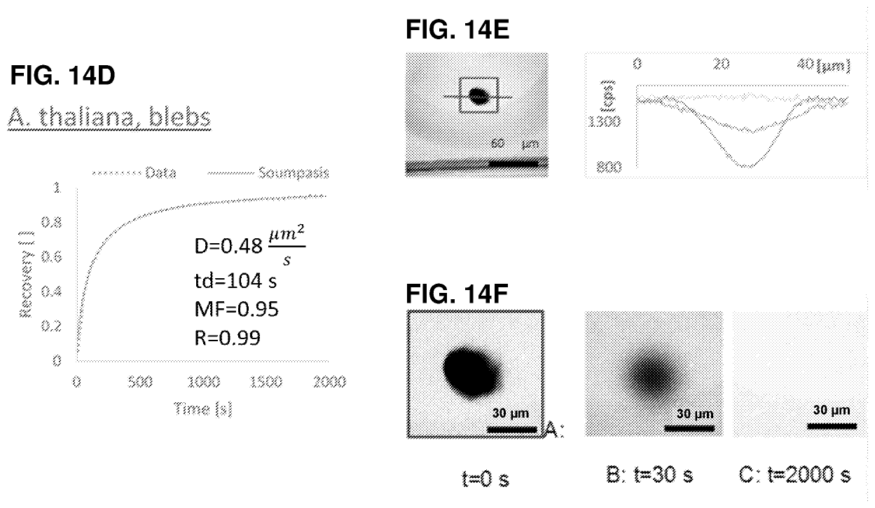

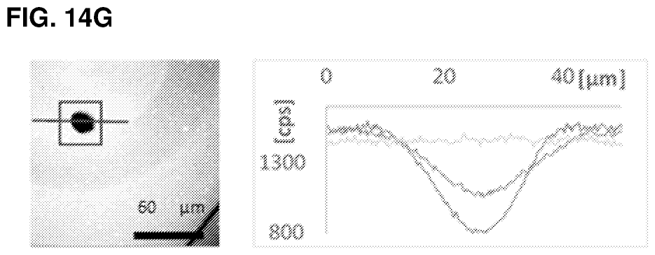

Formation of a supported lipid bilayer was confirmed using a rhodamine dye and fluorescent recovery after photobleaching (FRAP) (FIGS. 9A-9C, 13A-13C, and 14A-14G). For this approach, the plant-derived vesicles were deposited onto glass slides, ruptured using fusogenic vesicles, and imaged after 20 min incubation time. FIGS. 9A-9C show the results of a FRAP experiment for a supported lipid bilayer produced from N. benthamiana vesicles. FIGS. 13A-13C show the results of a FRAP experiment for a supported lipid bilayer produced from N. benthamiana membrane bubbles. FIG. 13A shows recovery of fluorescence at the bleached spot after 1200 seconds. FIG. 13B shows recovery of fluorescence after photobleaching for a supported lipid bilayer comprising N. benthamiana membrane bubbles. The experimental data has high alignment to a Soumpasis model. FIGS. 14A-14C show the results of a FRAP experiment for a supported lipid bilayer produced from A. thaliana membrane bubbles. FIGS. 14D-14F show the results of a FRAP experiment for a supported lipid bilayer produced from A. thaliana membrane blebs. FIG. 14A shows recovery of fluorescence after photobleaching for a supported lipid bilayer comprising Arabidopsis thaliana membrane bubbles. The experimental data has high alignment to a Soumpasis model (R=0.98). FIG. 14C shows complete recovery of fluorescence at the bleached spot after 2000 seconds. FIG. 14D shows recovery of fluorescence after photobleaching for a supported lipid bilayer comprising Arabidopsis thaliana membrane blebs. The experimental data has high alignment to a Soumpasis model (R=0.99). FIG. 14F shows complete recovery of fluorescence at the bleached spot after 2000 seconds.

These experiments demonstrated that lipid species are mobile and that the mobility is in a two-dimensional plane, indicating that a planar structure has been formed. Measuring the mobile fraction indicates the fraction of material that is mobile, confirming that the supported lipid bilayer is contiguous and well-healed.

c) Production of Protoplasts

Protoplasts were prepared from 3 week old Zea mays plants by enzymatic digestion of leaf tissue following the protocol of Richter et al., The Plant Cell, 28(10): 2651-2665, 2016. Plant material was bleached for 2 days by turning off the light in the growth chambers to reduce the chloroplast content. The middle section of each leaf was harvested. For dicots (N. benthamiana) the mid rib was removed, and the leaf blade was cut into 1 mm stick strips with a new razor blade, whereas only the middle part of the monocot (Zea mays) leaves was used to achieve more homogeneous protoplast results. The chopped material was submerged in digestion buffer (For 10 mL buffer, Cellulase “Onozuka” R-10 (Yacult Pharmaceutical, Japan) 0.15 mg (150 mg), Maceroenzyme Zacult (Yacult Pharmaceutical, Japan) 0.06 g (60 mg), Mannitol (MP Medicals) (0.6 M) 6 g (6 mL) (4 mL for tobacco, pH 5.8), MES (Sigma) 2 g (200 uL), CaCl (Sigma) 0.01 g (10 uL), 2-Mercaptoethanol (Sigma) 0.0105 (3 uL), Bovine serum albumin fraction V (Sigma) 0.01 g (100 mg)) filled up to 10 mL, and vacuum (80 kPa) was applied for 3.5 hours. The digested material was stirred gently with a spatula and filtered through a 100 μm nylon net. The protoplast-containing supernatant was centrifuged at 250 rcf for 2 min and the supernatant discarded. The pellet was resuspended in GPMVM buffer (2 mM CaCl2), 10 mM HEPES, 150 mM NaCl at pH 5.5, 0.2M Mannitol) and the washing step repeated two times. Plasma membrane vesicles (PMV) were collected from the digestion buffer and tested to contain sealed, right-side-out vesicles.

d) Production of Cell Plasma Membrane Blebs and Bubbles

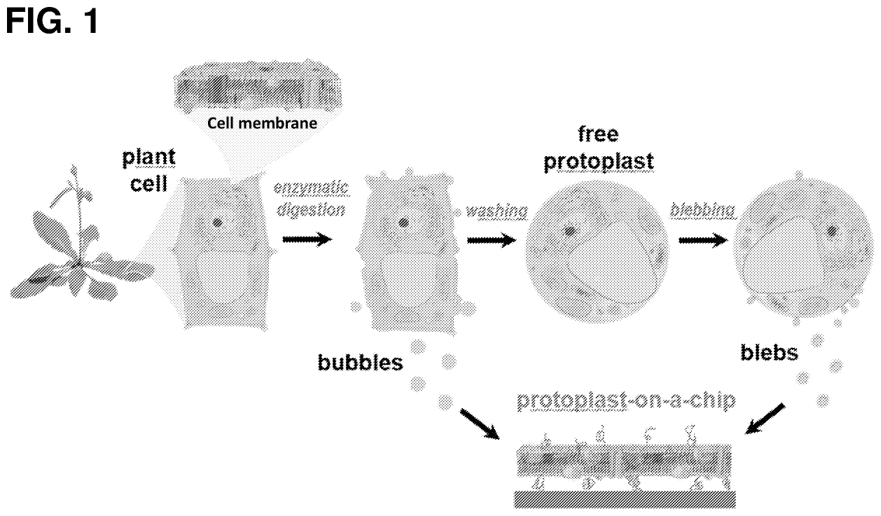

We generate small vesicles from these plant cells in two ways. First, during the dissolution of the cell wall, small vesicles named ‘bubbles’ are generated and collected from the supernatant. Second, we chemically treat the fully extracted protoplasts to induce blebbing of small protein-rich vesicles, named ‘blebs’. Either of these, bubbles or blebs, can be made to rupture onto a surface to self-assemble into a membrane sheet (a single bilayer) that conforms to the surface, but retains critical features of the membrane like lipid and protein two-dimensional mobility and rearrangement capabilities, proper orientation (outside facing up), and membrane heterogeneity and possibly sub-structures like lipid domains, etc. (FIG. 1).

To produce cell plasma membrane blebs, protoplasts were prepared from transgenic or transiently expressing leaf tissue as described above and were pelleted at 250 rcf for 2 min at 18° C. and resuspended in 4 mL blebbing buffer consisting of GPMVM buffer (2 mM CaCl₂), 10 mM HEPES, 150 mM NaCl at pH 7.4, 0.2 M Mannitol). 25 mM formaldehyde (FA) and 2 mM dithiothreitol (DTT) were included in the buffer to induce cell blebs. The cells were incubated in the blebbing solution for 3 hours at 23° C.

Cell plasma membrane blebs were also induced using osmotic stress (OS). In this method, cells were resuspended in 4 mL of GPMV buffer without Mannitol to keep the osmotic pressure constant, leading to a higher stress level, leading to vesicle budding and subsequent burst of the protoplasts. This mixture was also incubated for 3 h at 12° C.

The blebbing solutions containing cells and blebs were settled on ice for 30 min to separate cell debris, and cell blebs were collected from the supernatant.

Multiple different experiments covering a wide range of blebbing durations were tested. These experiment indicated that the formation and budding of blebs happens primarily within the first two hours and was not significantly higher after 24 h.

In contradiction to blebs, bubbles were generated passively during the digestion step. This approach uses the destabilizing conditions during cell wall digestion. During the digestion step, the formation and detachment of giant plasma vesicles during the initial steps of cell wall digestion were observed, wherein parts of the plasma membrane were pushed through holes in the primary cell wall. This procedure is probably facilitated by the negative osmotic pressure gradient from the cytosol to the buffer, which was approximately in the range of 3:1 (determined by a gradient dilution followed by counting of intact protoplasts). When first holes are digested in to the plant cell wall, the osmotic pressure is responsible to drive the formation of vesicles by diffusion of water into the cytoplasm and simultaneous swelling, which pushes parts of the plasma membrane into the exoplasmatic space, followed by a separation due to surface tension to pinch off the vesicle.

The digestion procedure is able to produce plasma membrane-derived vesicles which are able to bud off the protoplast by the combination of two essential factors. The first factor essential for the formation of bubbles is the cell wall. The cell wall is gradiently digested by mainly cellulase and pectinase, leaving behind holes in the structure. This process can be controlled by enzyme concentration and temperature and pH of the buffer solution. What usually comprises a big problem for the regular protoplast formation (as the protoplasts do not survive this gradient digestion) is beneficial for the induction of bubbles. The second important parameter and driving force for the formation of bubbles is the osmotic pressure difference. This is usually the major obstacle for the production of protoplasts but is utilized here to our advantage. The concentration of nutrients, carbohydrates and proteins solubilized in the cytosol are generating an osmotic pressure leading to the influx of apoplastic water into the cytosol. This water influx is referred in the literature as cell turgor and provides essential functions for the plant cell. This water influx would lead to the immediate bursting of the plant cell if it were not surrounded by the cell wall, providing a counter-pressure to stabilize the system. To conclude, in an environment with a lower osmotic pressure than the cytosol, the plant cell wall is protecting the plant well from bursting by physically restricting the expansion of the plasma membrane. The opposite of this would be the shrinking of the plant cell by a higher osmotic pressure at the outside of the cell plasma membrane. If the cell wall is partially digested in a lower osmotic environment, the stabilizing function which is necessary under this conditions to prevent the bursting probably leads to a swelling of the cytosol which is squeezed into the apoplastic space (outside the plant cell wall) which, when filled enough, buds off such as a “soap bubble”. The osmotic conditions are crucial for the success of this method. We want to create a slight overpressure to trigger the vesicle formation without rupturing them by the inflow of too much water. The osmotic pressure difference is mainly generated by the Mannitol in the digestion buffer. We found that 0.4 M Mannitol in the buffer is suitable for all the three plants (Arabidopsis thaliana, Nicotiana benthamiana, and Zea mays) and is suitable for other plants.

Alternatively, plant plasma membrane vesicles may be produced using a method comprising grinding a plant or a plant part (e.g., a leaf), e.g., submerging the plant or plant part in liquid nitrogen and grinding the plant or plant part in a buffer. Methods for grinding plant material, including appropriate buffers, are known in the art.

e) Characterization of Bleb and Bubble Size, Concentration, and Charge

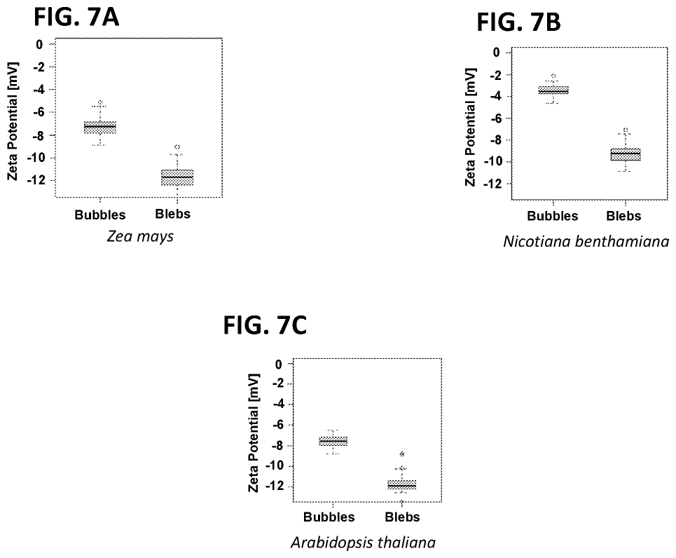

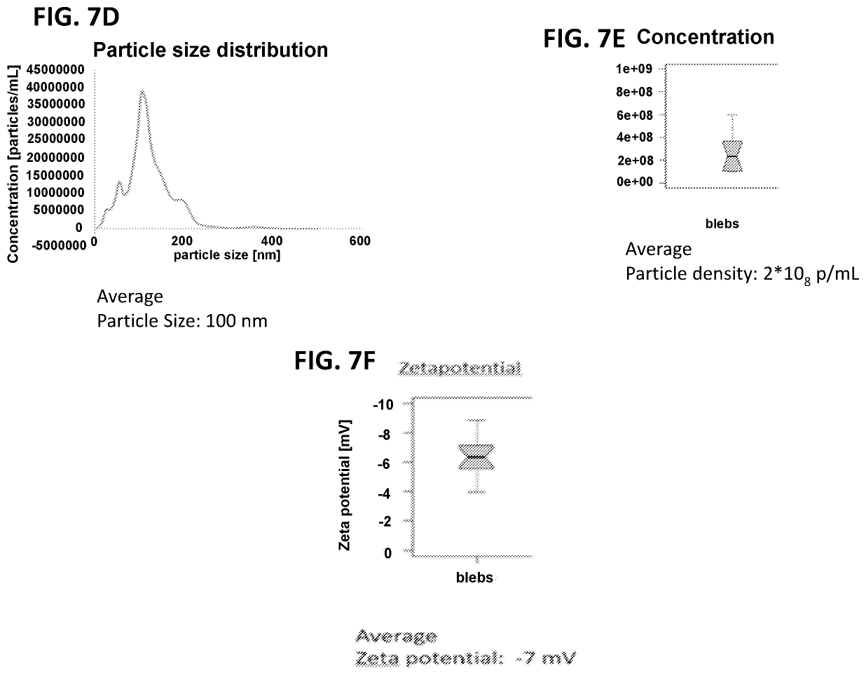

To determine the nanoparticle concentration and size distribution in the supernatant, Nanoparticle tracking analysis (NTA, Nanosight NS300, Malvern) was used. Dynamic Light Scattering and Electrophoretic Light Scattering (Zetasizer Nano ZS, Malvern) were used to measure the size distribution and charge of blebs and bubbles. All measurements were performed in GPMVM buffer at pH 5.5 at 23° C. For the Nanosight analysis, all measurements were analyzed using a detection threshold of 12 units. The resulting species were investigated by nanoparticle tracking analysis (NTA) and laser Doppler electrophoresis to assess their average size, zeta potential, and concentration. The data shows no significant difference for the zeta potentials of bubbles and blebs and an average value of about −7 mV (FIGS. 7A-7C). This result indicates that the blebbing buffer and the associated chemicals had no significant influence on the charge of the particles. The biggest influence on vesicle size was the clearing power of the centrifugation step, which showed obviously a negative correlation between relative centrifugal force and size. However, the size of blebs was slightly smaller and more homogeneous in average then the spontaneously formed bubbles. The typical size range of cell blebs is between 100-500 nm, with the average size for both vesicle species ranging from 100-200 nm (FIG. 7D). The concentrations of extracellular vesicles (e.g., bubbles or blebs) isolated from supernatant were consistently around 10⁷-10⁸ bubbles or blebs/ml (FIG. 7E). In summary, the average zeta potential was not significantly affected by the method.

Table 1 shows the average concentration, hydrodynamic diameter, and zeta potential of A. thaliana, N. benthamiana, and Zea mays bubbles and blebs.

TABLE 1
Vesicle characteristics

| | A. Thaliana | | N. benthamiana | | Z. mays | |
|---|---|---|---|---|---|---|
| | Bubbles | Blebs | Bubbles | Blebs | Bubbles | Blebs |
| Concentration | 4 × 10⁷ | 6 × 10⁷ | 5 × 10⁷ | 8 × 10⁵ | 5 × 10⁷ | 3 × 10⁶ |
| (p/mL) | | | | | | |
| Ave. hydrodynamic | 700 ± 80 | 450 ± 10 | 910 ± 20 | 520 ± 10 | 650 ± 60 | 640 ± 20 |
| diameter (nm) | | | | | | |
| Zeta potential (mV) | −7 ± 1 | −11 ± 2 | −8 ± 2 | −10 ± 1 | −7 ± 1 | −10 ± 2 |

f) Preparation of Liposomes

The following lipids were used in these experiments. 1-oleoyl-2-palmitoyl-sn-glycero-3-phosphocholine (POPC) and 1-oleoyl-2-palmitoyl-sn-glycero-3-phosphocholine (POPC-PEG), purchased from Avanti Polar Lipids. The liposomes were prepared by mixing the individual components in chloroform (Sigma) in a 99.5:0.5 ratio. Chloroform was gently evaporated using a stream of nitrogen, then the lipid films were stored under deep vacuum for 3 hours to remove any residual chloroform. To create liposomes, the GPMVM buffer was added to the dried films to reach a concentration of 2 mg/ml. Single unilamellar liposomes were prepared by high pressure extrusion (500 kPa) using a 50 nm membrane with at least 10 passes. The POPC-POPC-PEG (99.5% w/v POPC, 0.5% POPC-PEG) liposomes were used to rupture the plant cell plasma membrane blebs to form a continuous supported lipid bilayer. The blebs were labeled with R18 (Rhodamine dye) and functioned as gap filler for eventual irregularities in the membrane or separately when used as a control experiment for the FRAP experiment.

g) Plant Lipid Bilayer Formation on a Surface

All glass slides (25×25 mm, No. 1.5; VWR) were pretreated with a piranha solution (70% (v/v) H₂SO₄ (BDH) and 30% (v/v) H₂O₂ (Sigma 50 wt %)) for 10 min and rinsed by flushing DI water continuously for 20 min. To hold the sample, Polydimethylsiloxane (PDMS) wells were made by mixing 10:1 elastomer: cross linker mixture of Sylgard 184 (Robert McKeown Company) and baking for 2 h at 80° C.

Plasma membrane derived vesicles are inherently stable, can withstand a broad spectrum of osmotic pressure conditions, and generally do not rupture by themselves. In order to form a contiguous supported lipid bilayer on a planar surface, two different methods were used.

The first method used synthetic fusogenic lipid vesicles, which are zwitterionic in nature and rupture instantaneously once in contact with a negatively charged glass surface. These synthetic vesicles are used to trigger the rupture and facilitate the closing of isolated bilayer patches. Harvested blebs and bubbles were placed onto the glass slide and incubated (static) for 20 min. After incubation, the PDMS well was rinsed extensively with GPMVM buffer pH 5.5 and the POPC-PEG 5000 liposomes were added. The subsequent rupture process was observed under the microscope (data not shown) and took comparably longer than for vesicles derived from mammalian cells, but was usually completed within 30 min-90 min. A possible explanation for this relatively slow rupture is the high protein content in plant plasma membranes, which is also represented in the low diffusivity constant.

In some experiments, the blebs or bubbles were labeled with Rhodamine 18 (R18) at a concentration of 4 μL (1 mg/mL) per 1 mL blebs or bubbles and sonicated for 30 min. After sonication, the excess dye was removed by a size exclusion chromatography step, performed in Illustra MicroSpin G-25 Columns (GE Healthcare) at 300 rcf for 3 min. After PDMS wells were attached to the glass slides, 100 μL of flow through containing labeled bubbles/blebs at approximately 5×10⁷ blebs/mL was added into the well and incubated for 20 min static at room temperature. After incubation, the well was rinsed gently with GPMVM buffer to remove all unattached bubbles/blebs. 50 μL of liposomes (POPC-PEG 5000, produced as described in Example 1(f)) at a concentration of 2 mg/mL was added into the well and incubated for 30-3 h to form the supported bleb bilayer. After the bleb bilayer formed, the glass slides were scratched with a glass fragment and rinsed again with GPMVM buffer pH 5.5 to remove the excess liposomes.

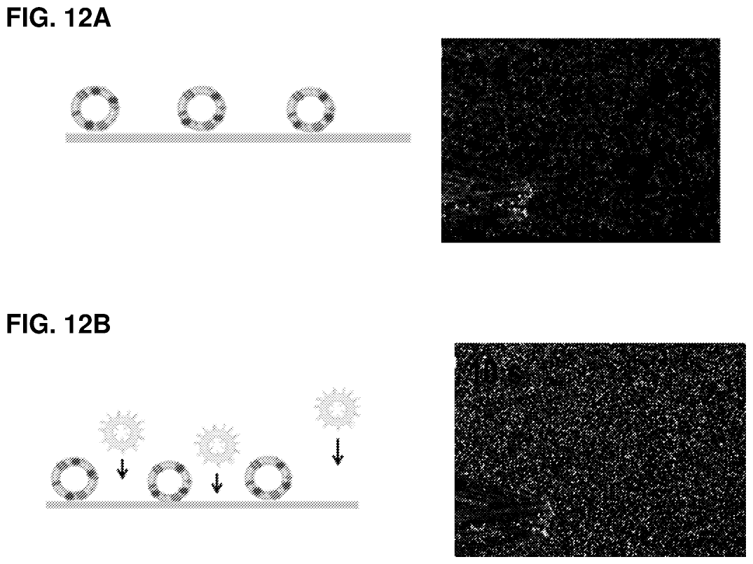

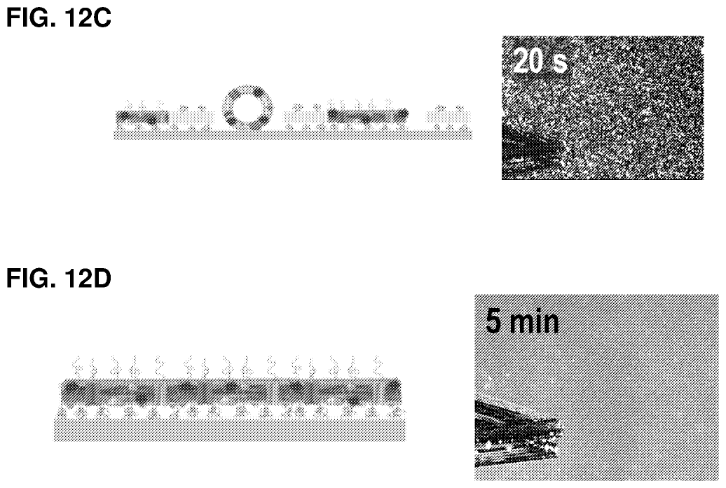

FIGS. 12A-12D show assembly of a supported plant plasma membrane comprising N. benthamiana blebs including the plasma membrane label Octadecyl Rhodamine B Chloride (R18). In FIG. 12A, R18-labeled Nicotiana benthamiana blebs (plant vesicles) are adsorbed to the surface, but have not ruptured. In FIG. 12B, fusogenic vesicles comprising PEG5K and DOPC have been added to the surface comprising R18-labeled N. benthamiana blebs. The R18 is dequenched, and fluorescent signals appear. In FIG. 12C, R18-labeled N. benthamiana blebs have ruptured in the presence of fusogenic vesicles comprising PEG5K and DOPC, and bilayer patches supported on the surface have formed. Some vesicles are still in the process of rupturing. In FIG. 12D, R18-labeled N. benthamiana blebs have ruptured in the presence of fusogenic vesicles comprising PEG5K and DOPC, and a continuous, homogenized lipid bilayer supported on the surface has formed.

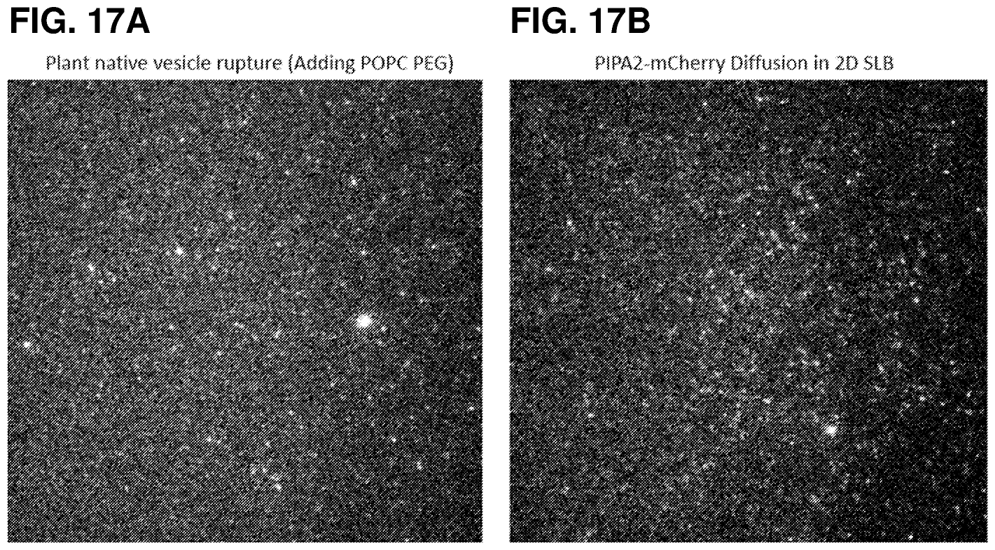

In some experiments, the blebs or bubbles included a fluorescent plasma membrane marker. FIGS. 17A and 17B show assembly of a supported plant plasma membrane comprising N. benthamiana blebs including the plasma membrane marker protein PIP2A-mCherry. FIG. 17A shows the PIP2A-mCherry fluorescent signal from intact cell blebs adsorbed on a glass slide. FIG. 17B shows the PIP2A-mCherry marker following the addition of POPC-PEG, rupture of the adsorbed blebs, and formation of a supported lipid bilayer, indicating that a protein from the blebs (i.e., PIP2A-mCherry) is successfully incorporated into the supported plant lipid bilayer.

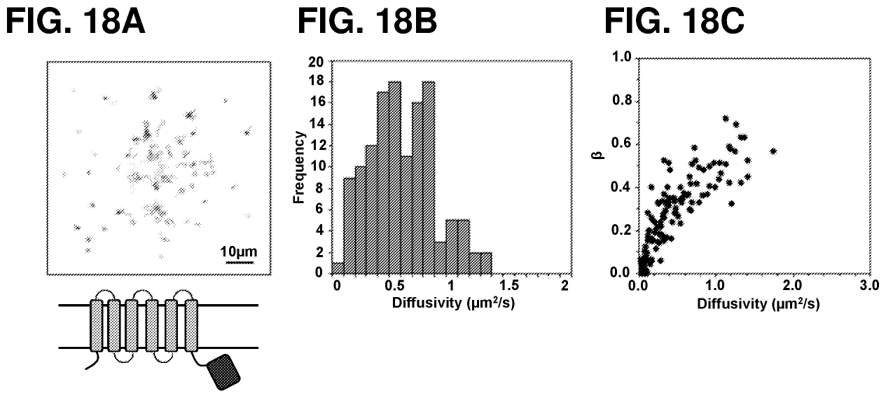

FIGS. 18A-18C show diffusion of single PIP2A-mCherry proteins in a supported plant lipid bilayer, indicating that the platform preserves the mobility and diffusivity of PIP2A-mCherry. These properties are essential for protein function. FIG. 18A shows trajectories of single PIP2A-mCherry proteins. FIGS. 18B and 18C show quantifications of the diffusivity of tracked particles.

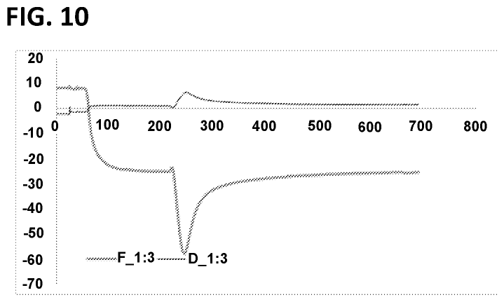

The mechanism of formation of the planar bleb bilayer was verified by quartz crystal microbalance (QCM-D) measurements and direct observation of membrane-bound fluorescent species diffusing from ruptured blebs (FIG. 10), as described below. As shown in FIG. 10, the QCM-D measurement shows the adsorption of blebs and the formation of a bilayer from blebs after the addition of liposomes (dissipation returns to ˜0).

h) Quartz Crystal Microbalance Analysis

Quartz Crystal Microbalance Combined with Dissipation Monitoring (QCM-D) was Used to quantify bilayer deposition as well as binding kinetics to the membrane. The measurement principle for QCM is a change in resonance frequency (Δf) and energy dissipation (ΔD) of a piezoelectric quartz crystal which is stimulated by an alternating current (AC) to oscillating in its eigenfrequency. Every change in frequency (Δf) or overtones reflects the change in adsorbed mass on the sensor surface. For these measurements, the third overtone frequency (15 MHz) was recorded during the experiment. Lower overtones were excluded from the data, as these appear to be rather instable due to edge effects (Hsia et al., Scientific Reports 6, 32715, 2016). Simultaneously, changes of energy dissipation (ΔD) were recorded to characterize the viscoelastic properties of the attached material. All experiments were measured on silicon dioxide (QSX303, Q-Sense) QCM-D crystals using a Q-Sense E1 instrument. Prior to the experiments, sensors were sonicated in a 3% [w/v] SDS solution for 30 min, cleaned with Milli-Q water, and dried with nitrogen gas. The clean sensors were then treated in an UV-Ozone Procleaner (Bioforce, USA) for 10 minutes to remove any organic contamination and to charge the surface of the silicon dioxide. Although the QCM sensors chosen here are composed of a similar material to the cover slips used in the microscopic experiments, the difference in elemental composition and surface texture can influence the bilayer formation kinetics, which should be taken into account when comparing the microscopy to the QCM-D results. Measurements were performed under constant flow conditions of 500 L/min pumped into the chamber by a peristaltic pump (Ismatec Reglo Digital M2-2/12, Q-Sense).

All experimental steps were performed at the same flow rate of 500 μL/min. For the control experiments, a simple PEG (5 k) 0.5% DOPC bilayer was formed. Therefore the QCM sensor was equilibrated with the buffer solution for 1 minute to determine the baseline (i.e. Δf=ΔD=0) of the sensor. Then the PEG (5 k) 0.5% DOPC liposomes were pumped into the chamber for about 3 minutes until a bilayer was formed and the values for Δf and AD stabilized. Then the system was rinsed with GPMVM buffer for 1 minute to wash out excess liposomes and to determine the final frequency and dissipation shifts.

Experiments were conducted in the same manner. The QCM sensor was equilibrated with the buffer solution for 1 minute to determine the baseline (i.e. Δf=ΔD=0) of the plain sensor. Then the cell-derived vesicles (blebs or bubbles) were sent into the chamber and circulated for about 3 minutes to attach to the sensor. Then the system was rinsed with GPMVM buffer for 1 minute to wash out excess vesicles, followed by the PEG (5 k) 0.5% DOPC liposomes which were pumped into the chamber for about 3 minutes until a stable supported lipid bilayer was formed and the values for Δf and AD reached a stable level. Finally, the system was rinsed with GPMVM buffer for 1 minute to wash out excess liposomes and determine the final values of frequency and dissipation. All experiments were recorded using Qsoft version 1.1.28.4, and normalized changes of frequency and dissipation of the third overtone were monitored and fit to a two-layer Voigt-Voinova viscoelastic model.

i) Single Particle Tracking Analysis

Fluorescently labeled transmembrane proteins were imaged using total internal reflection microscopy (TIRFM). The formed bilayers incorporating the protein of interest (PIN1-YFP, AtMate-MCherry, PIP2A-MCherry, or mCitrine-RCI2A) were recorded at the settings described below. Various methods for single particle tracking (SPT) have already been described in previous literature (Vrljic et al., Lipid Rafts, 193-219, 2007; Sonnleitner et al., Biophysical Journal, 77(5), 2638-2642, 1999; Poudel et al., Lipid-Protein Interactions: Methods and Protocols, 233-252, 2013; Ferrari et al., Physica D: Nonlinear Phenomena, 154(1), 111-137, 2001).

To enable accurate tracking, all trajectories were calculated by using the single particle tracking method previously reported in the literature (Richards et al., Langmuir, 32(12): 2963-2974). In order to calculate the trajectory of a migrating particle in a supported lipid bilayer (simplified as a 2D space), the SPT algorithm uses various sets of information gained from image analysis such as intensity, displacement and variability of intensity throughout multiple frames (Smith et al., Biophysical Journal, 101(7): 1794-1804, 2011). For a particle, three different kinds of motion are possible: confined motion, 2D planar motion, and 3D motion. If the particle is mobile in an area smaller than the maximum observed displacement for an equivalent immobile fluorescent bead, then the system is regarded as immobile (Sbalzarini et al., Journal of Structural Biology, 151(2), 182-195, 2005). The lipid and protein composition of the PM is highly heterogeneous, and lipid-to-protein mass ratio in the plant PM is approximately 1:1. To deal with this heterogeneous system, the single particle tracking algorithm uses the slope of the mean squared displacement (MSD) of the first three frames to determine the local “homogenous” diffusion coefficient (Smith et al., Biophysical Journal, 76(6): 3331-3344, 1999; Kusumi et al., Biophysical Journal, 65(5): 2021-2040, 1993). By doing so, the heterogeneity of the different domains of the bilayer does not strongly influence the initial diffusion coefficient. To take account for this heterogeneity, Ferrari et al. (Ferrari et al., Physica D: Nonlinear Phenomena, 154(1), 111-137, 2001; Sbalzarini et al., Journal of Structural Biology, 151(2), 182-195, 2005) employ moment scaling spectrum (MSS) analysis. This method utilizes a parameter β to characterize the motion of a molecule as one of three states: β<0.4 is confined diffusion; 0.4≤β≤0.6 is quasi-free diffusion; and β>0.6 is convective diffusion. All of the data was analyzed with Matlab and ImageJ (FIJI).

The mobility of proteins is essential for a biomimetic bilayer and has been one of the major obstacles in the production of a biomimetic platform. Different approaches, including pegylated liposomes and PEG cushions, have been used to address this problem. The nonspecific crosslinking effect of formaldehyde might affect proteins and phase partitioning at higher concentrations, but has no significant effect at the concentrations used (Fraenkel-Conrat and Olcott, Journal of the American Chemical Society, 70(8): 2673-2684, 1948; Sezgin et al., Nature Protocols, 7(6), 1042-1051, 2012; Levental, et al., Proceedings of the National Academy of Sciences, 107(51), 22050-22054, 2010).

Table 2 shows diffusivity and mobility of the membrane probe R18 in supported lipid bilayers derived from A. thaliana, N. benthamiana, and Zea mays bubbles and blebs.

TABLE 2
Diffusion and mobility of membrane probe R18

| | A. Thaliana | | N. benthamiana | | Z. mays | |
|---|---|---|---|---|---|---|
| | Bubbles | Blebs | Bubbles | Blebs | Bubbles | Blebs |
| Diffusion | 0.44 ± 0.11 | 0.48 ± 0.14 | 0.35 ± 0.08 | 0.15 ± 0.16 | 0.35 ± 0.17 | 0.35 ± 0.15 |
| Coefficient [μm²/s] | | | | | | |
| Mobile percent [%] | 98 ± 2 | 95 ± 2.1 | 94 ± 1.8 | 94 ± 2.2 | 95 ± 2.2 | 95 ± 2.8 |

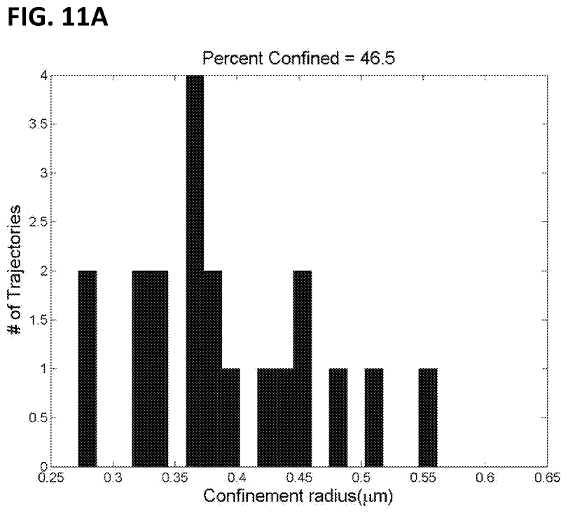

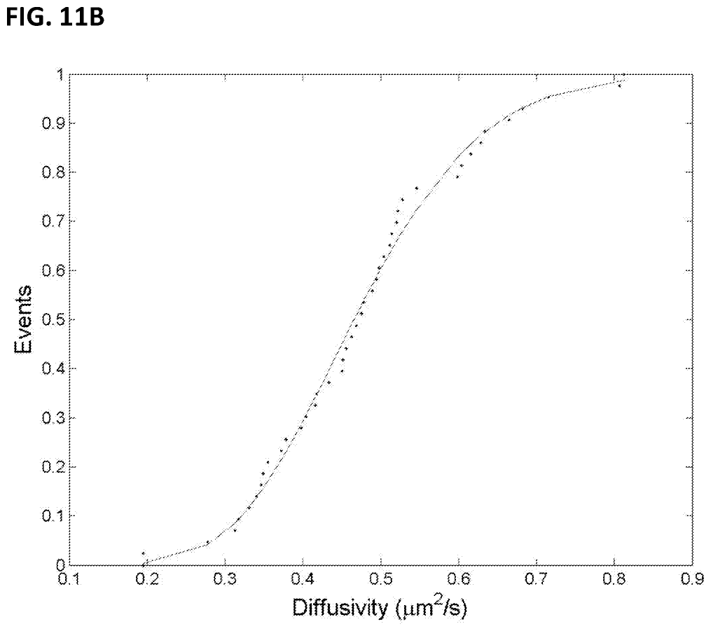

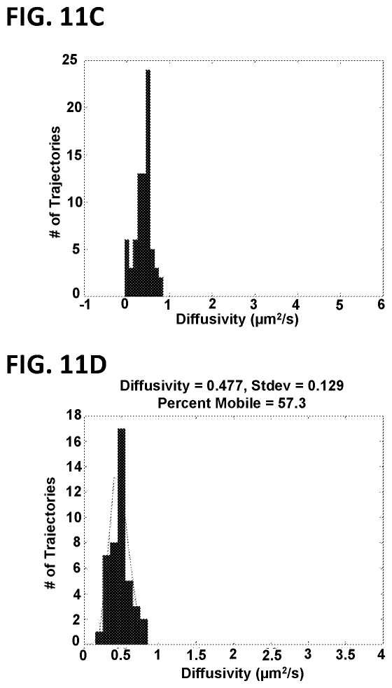

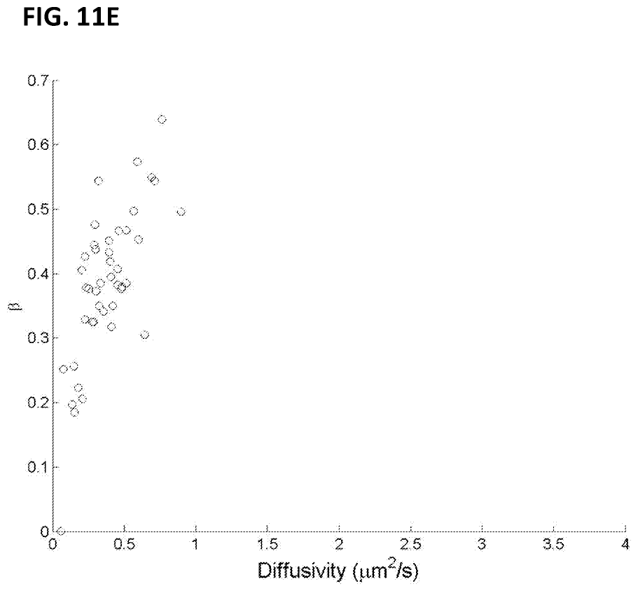

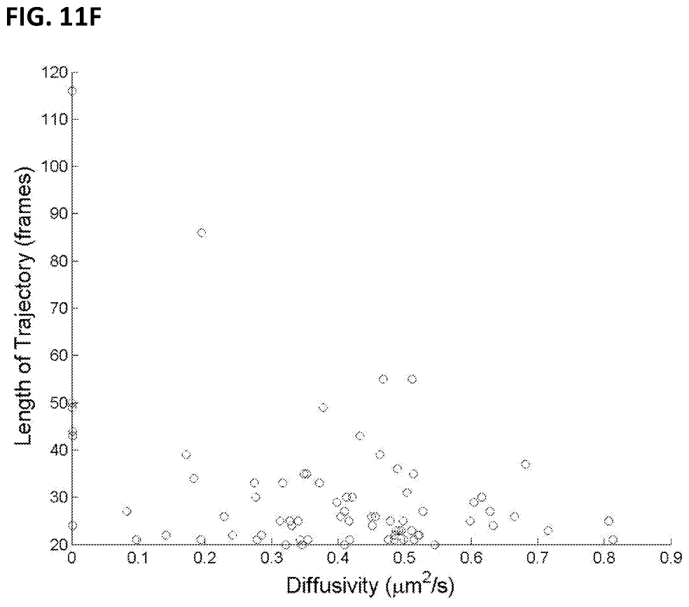

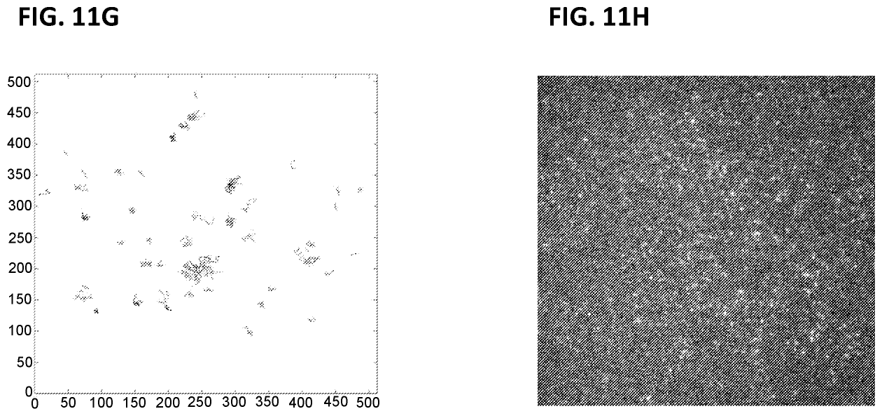

FIGS. 11A-11I show mobility, diffusivity, confinement, and tracked paths of single AtMate::YFP proteins. The results indicate that membrane proteins from native plant membrane material can be incorporated into a supported plant lipid bilayer and the platform can preserve the mobility of these membrane species, which is important for biological function.

Table 3 shows the mobility, diffusivity, and confinement of the tagged membrane protein PIP2A-mCherry in a supported lipid bilayer comprising vesicles from Nicotiana benthamiana.

TABLE 3
Single particle tracking analysis of fluorescently labeled membrane proteins

| Plant | MP | Diffusivity(μm²/s) | Mobility(%) | Confinement(%) |
|---|---|---|---|---|
| Nicotiana | PIPA2-mCherry | 0.631 ± 0.323 | 72.7 | 71.1 |
| benthamiana | | | | |
| Arabidopsis thaliana | RC12A-mCitrine | 0.726 ± 0.250 | 85.1 | 60.8 |
| Zea mays | PIN1-YFP | 0.477 ± 0.129 | 57.3 | 46.5 |

j) Characterization of Protein Expression

The presence of a protein of interest in the cell blebs was determined by Western blotting. In preparation for Western blotting, the cell bubbles/blebs were pelleted by an ultracentrifugation step (20,000×g for 1 h) and resuspended in 1 mL of GPMVM buffer. The bleb concentration was measured by Nanosight before and after the ultracentrifugation step to make sure that the sample was intact. The resuspended pellet was mixed with 2× Laemmli sample buffer (Bio-Rad) plus 10% DTT and incubated at 50° C. for 10 min. This step was followed by a separation on a SDS-PAGE (Bio-Rad) and transfer to a PVDF membrane (Millipore). The membrane was blocked with 5% dry milk in phosphate-buffered saline plus 0.01% Tween-20 (TBST) at room temperature for 1 h and subsequently incubated with antibodies against the protein of interest (1:1000, Everest Biotech) at room temperature.

k) Cryo-SEM Characterization

Standard conditions were used to generate the images of the vesicles (bubbles or blebs). Blebs were collected at the end of digestion; bubbles were collected by immersing the sample in liquid nitrogen 1 hour after the start of digestion. The sample was kept in the sublimation chamber for 5 min at 2*10\hat{ }6 torr and −100° C., and afterwards coated with a 2 nm thick gold-palladium layer at 10 mA for 10 s. This step is necessary for biological samples in order to avoid charging of the surface and further imaging artifacts. Images were obtained by detecting secondary electrons at 2*10\hat{ }6 torr and −165° C. using 5 kV electron beam currency. As the sample surface at 10k× magnification was already eroded after one second, most of the images were obtained in blind-zoom mode using a new spot for every image. After obtaining the primary image, a second image was generated using a much higher exposure time to observe the sample erosion patterns. This method was useful to distinguish between physical samples and imaging artefacts, as only those pictures were used that showed an unspoiled surface after the longer bombardment.

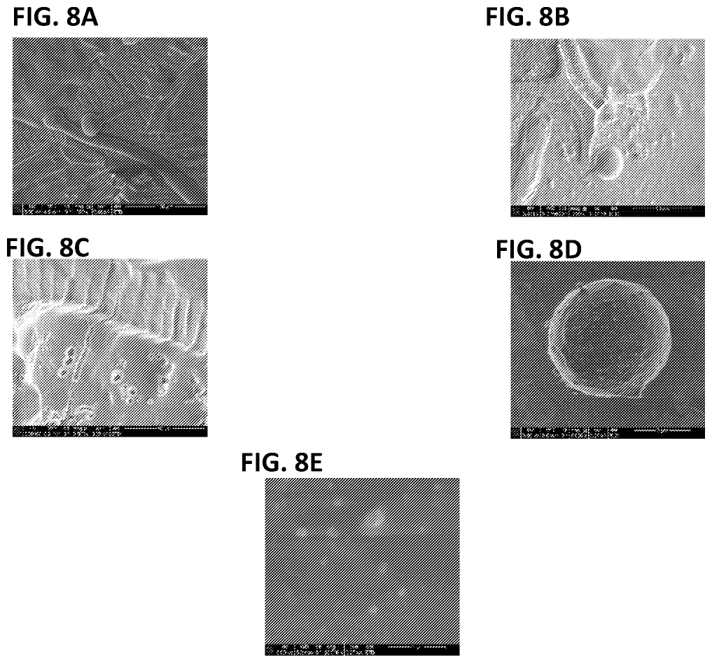

For imaging of bubbles, due to the crude cell lysate used for the bubble production, the imaging was mainly performed in native cracks that are obtained by the freezing process. We were able to capture the full digestion process, starting at full cell wall fragments down to the 400 nm bubbles (FIGS. 8A-8E).

The formation of blebs was also observed 20 min after transferring the purified protoplasts into the blebbing solution. The blebbing conditions had previously been optimized by Liu (Liu et al., ACS Applied Materials & Interfaces, 9(41): 35526-35538, 2017) for mammalian cells. By modifying this protocol, we optimized the conditions for plant cells using dynamic light scattering and maximizing the fraction between 200 nm and 800 nm.

l) Shelf Life of Bubbles

A sample comprising bubbles in the above-described digestion buffer containing the enzymes was stored at 5° C. for a week, labeled, and used to produce a supported plasma membrane. The experiment resulted in a smooth lipid bilayer, indicating that bubbles may be stored in the digestion buffer.

m) Uses

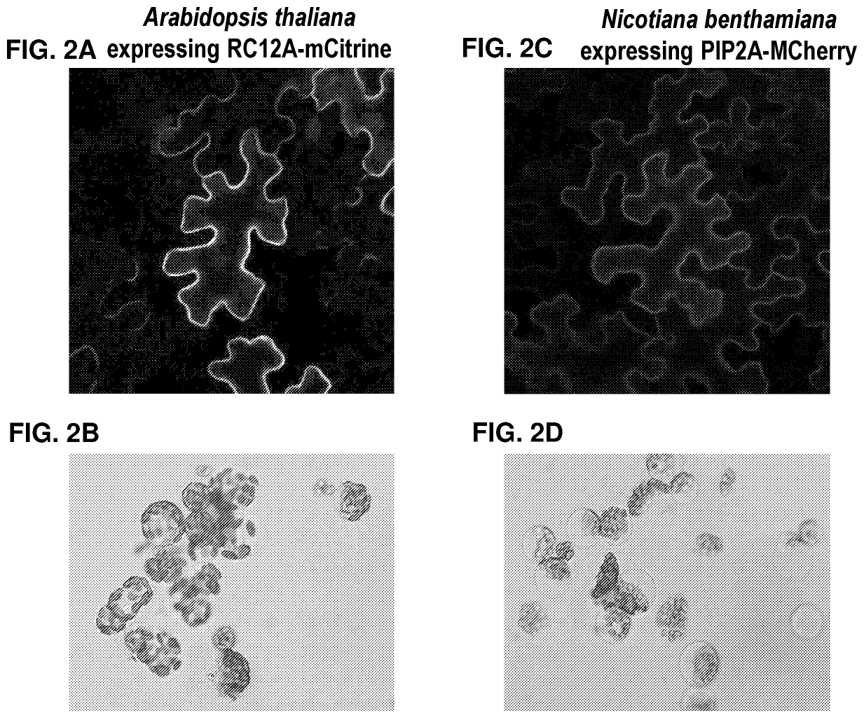

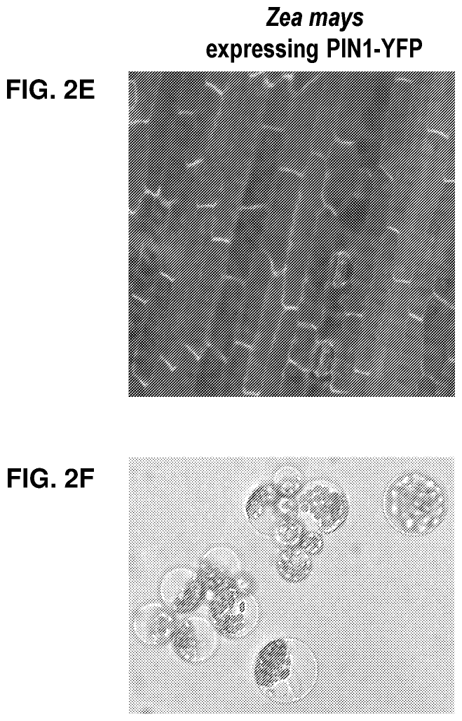

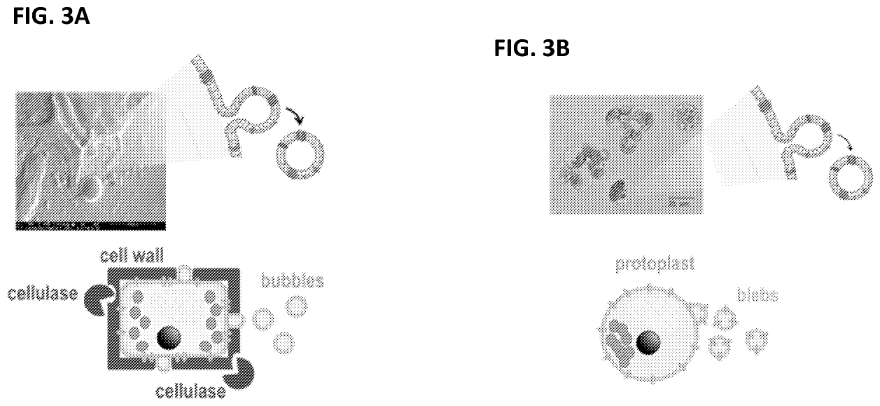

We have expressed fluorescently tagged membrane proteins into plant cells (FIGS. 2A, 2C, and 2E) and subsequently collected them in the bubbles and blebs (FIGS. 3A and 3B).

We have characterized the membranes formed on solid surfaces from these materials as well and shown their basic properties.

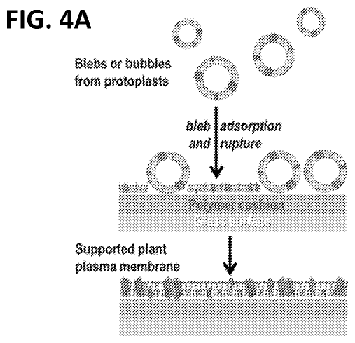

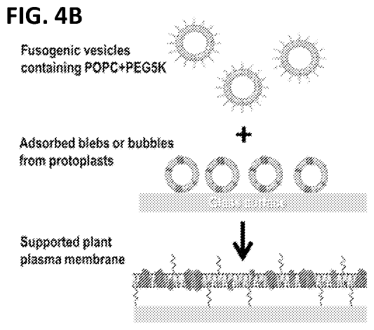

Membranes are self-assembled on the surface in two ways. First, with modification of the surface with attractive moieties to promote bubble or bleb rupture, such as polymers or other polyelectrolytes or protein layers, the bubbles and blebs rupture directly on the surface and heal together to create a contiguous membrane bilayer sheet. Second, bubbles or blebs can be adsorbed unruptured to a surface, such as unmodified glass or silica, and then induced to rupture by the addition of lipid-rich vesicles that readily rupture themselves to form bilayers on these surfaces. For example, POPC or POPC+PEGylated lipids. In the latter case, the PEGylated vesicles also promote a bigger space between the membrane sheet that forms and the surface, helping to promote protein mobility (FIGS. 4A and 4B).

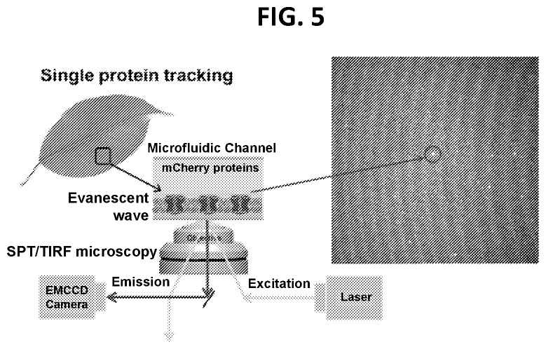

Proteins that are fluorescently tagged can be expressed in the protoplast cell membrane and harvested with the blebs. Following the procedure above leads to the introduction of these tagged proteins into the membrane sheet and the ability to monitor their motion and track their diffusion patterns (FIG. 5).

The procedures described are reliable ways to make the membrane sheets with bubbles or blebs. The most reliable surfaces to date has been glass, and other surfaces such as polyelectrolytes, PMETAC, PEDOT:PSS, PEG, PLL, cellulose.

The methods developed here should extend to surfaces of other geometries like spherical particles, or surfaces with corrugated structures or other surface features.

This approach can also be extended from the protoplasts to the chloroplasts (an internal plant organelle with membranes) to create a chloroplast platform.

In this study, we explored the possibilities of plant cell membrane derived blebs and bubbles, which reveals the effects of the conditions used to induce blebbing on the protein, lipid, and proteoliposome properties. The results suggest that both induction methods impact the protein behavior and vesicle formation differently

### Other Embodiments

Some embodiments of the invention are within the following numbered paragraphs.

1. A composition comprising a plant plasma membrane supported on a surface of an object, wherein the plant plasma membrane is not associated with a plant cell.

2. The composition of paragraph 1, wherein the plant plasma membrane is a lipid bilayer.

3. The composition of paragraph 1, wherein the plant plasma membrane is adsorbed to the surface of the object.

4. The composition of paragraph 1, wherein the plant plasma membrane adheres to the surface of the object.

5. The composition of paragraph 1, wherein the plant plasma membrane is derived from a cell plasma membrane.

6. The composition of paragraph 1, wherein the plant plasma membrane is derived from a chloroplast membrane.

7. The composition of paragraph 6, wherein the chloroplast membrane is a thylakoid membrane.

8. The composition of paragraph 1, wherein the plant plasma membrane is derived from a leaf, stem, or root of a plant.

9. The composition of paragraph 1, wherein the plant is a maize plant, an Arabidopsis plant, or a tobacco plant.

10. The composition of paragraph 1, wherein the plant plasma membrane has a protein and lipid orientation, mobility, and activity that is native to the plant plasma membrane.

11. The composition of paragraph 1, wherein the plant plasma membrane is modified.

12. The composition of paragraph 11, wherein the plant plasma membrane is derived from a plant or plant part that has been modified.

13. The composition of paragraph 12, wherein the modification comprises addition of a membrane component.

14. The composition of paragraph 13, wherein the membrane component is a lipid.

15. The composition of paragraph 13, wherein the membrane component is an exogenous protein.

16. The composition of paragraph 15, wherein the exogenous protein comprises a fluorescent marker.

17. The composition of paragraph 1, wherein the plant plasma membrane is produced by a method comprising blebbing.

18. The composition of paragraph 1, wherein the plant plasma membrane is produced by a method comprising bubbles.

19. The composition of paragraph 1, wherein the object is a glass microscope slide, a silicon wafer, a microfluidic channel, or a silica bead.

20. The composition of paragraph 1, wherein the surface is hydrophilic.

21. The composition of paragraph 1, wherein the surface comprises a coating.

22. The composition of paragraph 21, wherein the coating is a polymer.

23. The composition of paragraph 22, wherein the polymer is PMETAC.

24. The composition of paragraph 1, wherein the surface is chemically modified.

25. The composition of paragraph 1, wherein the surface of the object is flat.

26. The composition of paragraph 1, wherein the surface of the object is round, curved, corrugated, or possesses another geometric shape or topography.

27. A method for manufacturing a plant plasma membrane supported on a surface of an object, the method comprising:
(a) providing a plurality of vesicles, wherein each vesicle comprises an isolated plant plasma membrane; and
(b) contacting the plurality of vesicles with a surface of an object, wherein the isolated plant plasma membranes assemble into a continuous lipid bilayer, thereby forming the plant plasma membrane supported on the surface of the object.

28. The method of paragraph 27, wherein the plurality of vesicles is a plurality of bubbles and wherein the providing comprises treating a plant or plant part to produce a plurality of bubbles.

29. The method of paragraph 28, wherein the treating comprises administering an agent that disrupts a cell wall of the plant or plant part.

30. The method of paragraph 29, wherein the agent is a cellulase or a pectinase.

31. The method of paragraph 29, wherein the agent comprises a cellulase and a pectinase.

32. The method of paragraph 28, wherein the plant or plant part is a maize plant or plant part, an Arabidopsis plant or plant part, or a tobacco plant or plant part.

33. The method of paragraph 28, wherein the plant plasma membrane is a lipid bilayer.

34. The method of paragraph 28, wherein the plasma membrane is derived from a cell plasma membrane.

35. The method of paragraph 28, wherein the plant part is a chloroplast.

36. The method of paragraph 35, wherein the plasma membrane is a chloroplast membrane (e.g., a thylakoid membrane).

37. The method of paragraph 27, wherein the plurality of vesicles is a plurality of blebs and wherein the providing comprises:
(a) providing a plant protoplast; and
(b) treating the plant protoplast to produce a plurality of blebs.

38. The method of paragraph 37, wherein the providing of step (a) comprises administering an agent that disrupts a cell wall of the plant or plant part to produce a protoplast.

39. The method of paragraph 37, wherein the treating of step (b) comprises administering a blebbing buffer.

40. The method of paragraph 37, wherein the treating of step (b) comprises inducing osmotic stress.

41. The method of paragraph 37, wherein the protoplast is a maize protoplast, an Arabidopsis protoplast, or a tobacco protoplast.

42. The method of paragraph 37, wherein the plant plasma membrane is a lipid bilayer.

43. The method of paragraph 37, wherein the plasma membrane is derived from a cell plasma membrane.

44. The method of paragraph 27, wherein the plurality of vesicles is a plurality of bubbles and blebs and wherein the providing comprises:
(a) treating a plant or plant part to produce a plurality of bubbles;
(b) treating the plant or plant part of step (a) to produce a plant protoplast; and
(c) treating the plant protoplast of step (b) to produce a plurality of blebs.

45. The method of paragraph 27, further comprising modifying the plant plasma membrane.

46. The method of paragraph 27, wherein the contacting comprises causing the plurality of vesicles to rupture on a surface, wherein the ruptured vesicles create a continuous lipid bilayer.

47. The method of paragraph 46, wherein causing the plurality of vesicles to rupture comprises providing a surface comprising one or more attractive moieties.

48. The method of paragraph 47, wherein the attractive moiety is comprised by a coating on the surface.

49. The method of paragraph 48, wherein the coating is a polymer.

50. The method of paragraph 49, wherein the polymer is PMETAC.

51. The method of paragraph 47, wherein the attractive moiety is comprised by a chemical modification on the surface.

52. The method of paragraph 46, wherein the plurality of vesicles rupture directly on the surface.

53. The method of paragraph 27, wherein the surface does not comprise an attractive moiety.

54. The method of paragraph 53, wherein the plurality of vesicles are adsorbed unruptured to the surface.

55. The method of paragraph 46, wherein causing the plurality of vesicles to rupture comprises contacting the plurality of vesicles with a plurality of lipid-rich vesicles.

56. The method of paragraph 55, wherein the plurality of lipid-rich vesicles comprises vesicles comprising POPC or POPC-PEG.

57. The method of paragraph 54, wherein the surface comprises a coating or a chemical modification.

58. The method of paragraph 27, wherein the object is a glass microscope slide, a silicon wafer, a microfluidic channel, or a silica bead.

59. The method of paragraph 27, wherein the surface of the object is flat.

60. The method of paragraph 27, wherein the surface of the object is round, curved, corrugated, or possesses another geometric shape or topography.

61. The method of paragraph 27, further comprising separating the vesicles from one or more contaminants.

62. The method of paragraph 27, wherein the lipid bilayer comprises isolated plant plasma membranes derived from bubbles and blebs.

63. The method of paragraph 27, wherein the method does not comprise use of a detergent.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

What is claimed is:

1. A composition comprising a plant plasma membrane supported on a surface of an object, wherein the plant plasma membrane is not associated with a plant cell.

2. The composition of claim 1, wherein the plant plasma membrane is a lipid bilayer.

3. The composition of claim 1, wherein the plant plasma membrane is adsorbed to the surface of the object.

4. The composition of claim 1, wherein the plant plasma membrane adheres to the surface of the object.

5. The composition of claim 1, wherein the plant plasma membrane is derived from a cell plasma membrane.

6. The composition of claim 1, wherein the plant plasma membrane is derived from a chloroplast membrane.

7. The composition of claim 6, wherein the chloroplast membrane is a thylakoid membrane.

8. The composition of claim 1, wherein the plant plasma membrane is derived from a leaf, stem, or root of a plant.

9. The composition of claim 1, wherein the plant is a maize plant, an Arabidopsis plant, or a tobacco plant.

10. The composition of claim 1, wherein the plant plasma membrane has a protein and lipid orientation, mobility, and activity that is native to the plant plasma membrane.

11. The composition of claim 1, wherein the plant plasma membrane is modified.

12. The composition of claim 11, wherein the plant plasma membrane is derived from a plant or plant part that has been modified.

13. The composition of claim 12, wherein the modification comprises addition of a membrane component.

14. The composition of claim 13, wherein the membrane component is a lipid.

15. The composition of claim 13, wherein the membrane component is an exogenous protein.

16. The composition of claim 15, wherein the exogenous protein comprises a fluorescent marker.

17. The composition of claim 1, wherein the plant plasma membrane is produced by a method comprising blebbing.

18. The composition of claim 1, wherein the plant plasma membrane is produced by a method comprising bubbles.

19. The composition of claim 1, wherein the object is a glass microscope slide, a silicon wafer, a microfluidic channel, or a silica bead.

20. The composition of claim 1, wherein the surface is hydrophilic.

21. The composition of claim 1, wherein the surface comprises a coating.

22. The composition of claim 21, wherein the coating is a polymer.

23. The composition of claim 22, wherein the polymer is PMETAC.

24. The composition of claim 1, wherein the surface is chemically modified.

25. The composition of claim 1, wherein the surface of the object is flat.

26. The composition of claim 1, wherein the surface of the object is round, curved, corrugated, or possesses another geometric shape or topography.

27. A method for manufacturing a plant plasma membrane supported on a surface of an object, the method comprising:
(a) providing a plurality of vesicles, wherein each vesicle comprises an isolated plant plasma membrane; and
(b) contacting the plurality of vesicles with a surface of an object, wherein the isolated plant plasma membranes assemble into a continuous lipid bilayer, thereby forming the plant plasma membrane supported on the surface of the object.

28. The method of claim 27, wherein the plurality of vesicles is a plurality of bubbles and wherein the providing comprises treating a plant or plant part to produce a plurality of bubbles.

29. The method of claim 28, wherein the treating comprises administering an agent that disrupts a cell wall of the plant or plant part.

30. The method of claim 29, wherein the agent is a cellulase or a pectinase.

31. The method of claim 29, wherein the agent comprises a cellulase and a pectinase.

32. The method of claim 28, wherein the plant or plant part is a maize plant or plant part, an Arabidopsis plant or plant part, or a tobacco plant or plant part.

33. The method of claim 28, wherein the plant plasma membrane is a lipid bilayer.

34. The method of claim 28, wherein the plasma membrane is derived from a cell plasma membrane.

35. The method of claim 28, wherein the plant part is a chloroplast.

36. The method of claim 35, wherein the plasma membrane is a chloroplast membrane (e.g., a thylakoid membrane).

37. The method of claim 27, wherein the plurality of vesicles is a plurality of blebs and wherein the providing comprises:
(a) providing a plant protoplast; and
(b) treating the plant protoplast to produce a plurality of blebs.

38. The method of claim 37, wherein the providing of step (a) comprises administering an agent that disrupts a cell wall of the plant or plant part to produce a protoplast.

39. The method of claim 37, wherein the treating of step (b) comprises administering a blebbing buffer.

40. The method of claim 37, wherein the treating of step (b) comprises inducing osmotic stress.

41. The method of claim 37, wherein the protoplast is a maize protoplast, an Arabidopsis protoplast, or a tobacco protoplast.

42. The method of claim 37, wherein the plant plasma membrane is a lipid bilayer.

43. The method of claim 37, wherein the plasma membrane is derived from a cell plasma membrane.

44. The method of claim 27, wherein the plurality of vesicles is a plurality of bubbles and blebs and wherein the providing comprises:
(d) treating a plant or plant part to produce a plurality of bubbles;
(e) treating the plant or plant part of step (a) to produce a plant protoplast; and
(f) treating the plant protoplast of step (b) to produce a plurality of blebs.

45. The method of claim 27, further comprising modifying the plant plasma membrane.

46. The method of claim 27, wherein the contacting comprises causing the plurality of vesicles to rupture on a surface, wherein the ruptured vesicles create a continuous lipid bilayer.

47. The method of claim 46, wherein causing the plurality of vesicles to rupture comprises providing a surface comprising one or more attractive moieties.

48. The method of claim 47, wherein the attractive moiety is comprised by a coating on the surface.

49. The method of claim 48, wherein the coating is a polymer.

50. The method of claim 49, wherein the polymer is PMETAC.

51. The method of claim 47, wherein the attractive moiety is comprised by a chemical modification on the surface.

52. The method of claim 46, wherein the plurality of vesicles rupture directly on the surface.

53. The method of claim 27, wherein the surface does not comprise an attractive moiety.

54. The method of claim 53, wherein the plurality of vesicles are adsorbed unruptured to the surface.

55. The method of claim 46, wherein causing the plurality of vesicles to rupture comprises contacting the plurality of vesicles with a plurality of lipid-rich vesicles.

56. The method of claim 55, wherein the plurality of lipid-rich vesicles comprises vesicles comprising POPC or POPC-PEG.

57. The method of claim 54, wherein the surface comprises a coating or a chemical modification.

58. The method of claim 27, wherein the object is a glass microscope slide, a silicon wafer, a microfluidic channel, or a silica bead.

59. The method of claim 27, wherein the surface of the object is flat.

60. The method of claim 27, wherein the surface of the object is round, curved, corrugated, or possesses another geometric shape or topography.

61. The method of claim 27, further comprising separating the vesicles from one or more contaminants.

62. The method of claim 27, wherein the lipid bilayer comprises isolated plant plasma membranes derived from bubbles and blebs.

63. The method of claim 27, wherein the method does not comprise use of a detergent.
